(19) United States
(12) Patent Application Publication
(10) Pub. No.: US 2026/0284199 A1
(43) Pub. Date: Sep. 24, 2026

(54) GLUCURONOXYLOMANNAN FOR USE IN CENTRAL NERVOUS SYSTEM DELIVERY

(71) Applicant: University of Florida Research Foundation, Incorporated, Gainesville, FL (US)

(72) Inventor: Luis R. MARTINEZ, Gainesville, FL (US)

(21) Appl. No.: 19/472,341

(22) PCT Filed: Apr. 17, 2024
(86) PCT No.: PCT/US2024/024870

§ 371 Date: Oct. 3, 2025

Related U.S. Application Data
(60) Provisional application No. 63/496,716, filed on Apr. 18, 2023.

(51) Int. Cl. A61K 47/36; A61K 9/00; A61K 38/16; A61P 25/00; C07K 16/28

(52) U.S. Cl. CPC A61K 47/36; A61K 9/0043; A61K 9/0085; A61K 38/16; A61P 25/00; C07K 16/2896

(57) ABSTRACT

Described are compositions comprising isolated GXM and methods of using the compositions for increasing permeability of the blood brain barrier in a subject or increasing delivery of a molecule to the central nervous system of a subject.

### CROSS-REFERENCE TO RELATED APPLICATIONS

This application claims the benefit of U.S. Provisional Application No. 63/496,716, filed Apr. 18, 2023, which is incorporated herein by reference.

### STATEMENT REGARDING FEDERALLY SPONSORED RESEARCH OR DEVELOPMENT

This invention was made with government support under Grant No. R01 AI145559 awarded by the National Institutes of Health. The government has certain rights in the invention.

### BACKGROUND

The encapsulated fungus Cryptococcus neoformans is the most common cause of fungal meningitis, with the highest rate of disease in patients with AIDS or immunosuppression. C. neoformans enters the human body via inhalation of infectious particles. C. neoformans capsular polysaccharide, in which the major component is glucuronoxylomannan (GXM), extensively accumulates in tissues and compromises host immune responses. C. neoformans travels from the lungs to the bloodstream and crosses to the brain via transcytosis, paracytosis, or inside of phagocytes using a “Trojan horse” mechanism. The fungus causes life-threatening meningoencephalitis with high mortality rates.

C. neoformans capsular GXM is released into tissues during infection where it accumulates and causes a range of adverse immunological defects (Lee S C et al. “Polysaccharide antigen in brain tissue of AIDS patients with cryptococcal meningitis.” Clin Infect Dis. 1996; 23(1): 194-5). Individuals with cryptococcal meningoencephalitis frequently suffer from the detrimental effects associated with increased intracranial pressure such as edema, hydrocephalus, and subarachnoid space hemorrhage, which may be related in part to GXM-mediated effects (Hamed M F et al. “Clinical and pathological characterization of Central Nervous System cryptococcosis in an experimental mouse model of stereotaxic intracerebral infection.” PLOS Negl Trop Dis. 2023; 17(1): e0011068). The concentration of GXM in CSF can reach 1 mg/mL (Eng R et al. “The course of cryptococcal capsular polysaccharide antigenemia/human cryptococcal polysaccharide elimination kinetics. Infection. 1983; 11(3): 132-6.) and post-mortem pathological examinations of brains from patients with cryptococcal meningoencephalitis indicate that this polysaccharide is extensively deposited in tissue.

### SUMMARY

Described are methods of delivering or increasing delivery of a molecule to the central nervous system (CNS) of a subject comprising administering the molecule to the subject in combination with isolated glucuronoxylomannan (GXM). The molecule and the isolated GXM can be administered to the subject together (e.g., in a single composition or formulation) or separately (e.g., in separate formulations, e.g., a pharmaceutical combination). In some embodiments, the isolated GXM can be administered to the subject intranasally. In some embodiments, the isolated GXM can be administered to the subject intravascularly (e.g., intravenously). In some embodiments, the molecule is administered intranasally, intravascularly, intravenously, or subcutaneously. In some embodiments, delivering the molecule to the CNS comprises delivering the molecule to the brain. In some embodiments, the subject has a neurological disease, a CNS cancer, or a CNS infection. The molecule can be, but is not limited to, a therapeutic molecule (i.e., a pharmaceutical agent or active pharmaceutical ingredient). The therapeutic molecule can be, but is not limited to, a protein, an antibody, an antibody fragment, or a small molecule drug.

In some embodiments, the isolated GXM is administered to a subject at a concentration of about 5 μg/mL to about 200 μg/mL.

In some embodiments, the isolated GXM is isolated from C. neoformans. In some embodiments, the isolated GXM is microbe-free. In some embodiments, the isolated GXM comprises a cell-free preparation of GXM. In some embodiments, the isolated GXM is essentially pure. In some embodiments, the isolated GXM is at least 60%, at least 65%, at least 70%, at least 75%, at least 80%, at least 85%, at least 90%, or at least 95% pure.

Described are methods of increasing permeability of the blood brain barrier in a subject comprising administering to the subject isolated GXM. In some embodiments, the isolated GXM is administered to the subject by intranasal administration. In some embodiments, the isolated GXM is administered to the subject by intravascular administration. In some embodiments, the isolated GXM is administered to the subject by intravenous administration. In some embodiments, the isolated GXM is administered to a subject at a concentration of about 5 μg/mL to about 200 μg/mL. In some embodiments, the isolated GXM is isolated from C. neoformans. In some embodiments, the isolated GXM is microbe-free. In some embodiments, the isolated GXM comprises a cell-free preparation of GXM. In some embodiments, the isolated GXM is essentially pure. In some embodiments, the isolated GXM is at least 60%, at least 65%, at least 70%, at least 75%, at least 80%, at least 85%, at least 90%, or at least 95% pure.

In some embodiments, permeability of the blood brain barrier is increased transiently.

In some embodiments, increasing permeability of the blood brain barrier comprises one or more of: (a) altering the distribution and/or number of endothelial cell tight junctions; (b) altering endothelial cell tight junction protein expression; (c) reducing trans-endothelial electrical resistance of endothelial cells; (d) increasing Ras homolog family member A (RhoA) expression in endothelial cells; (e) increasing RhoA activation in endothelial cells; (f) increasing myosin light chain (MLC) phosphorylation in endothelial cells; (g) forming actin stress fibers in endothelial cells; (h) remodeling the cytoskeleton in endothelial cells; (i) increasing blood vessel relaxation; and (j) weakening brain endothelial cell/pericyte interactions. In some embodiments, altering endothelial cell tight junction protein expression comprises decreasing claudin-5 and/or occludin expression.

Described are compositions for delivery of a molecule to the CNS comprising the molecule and a pharmaceutically effective dose of isolated GXM. The molecule can be, but is not limited to, a therapeutic molecule (i.e., a pharmaceutical agent or active pharmaceutical ingredient). The therapeutic molecule can be, but is not limited to, a protein, an antibody, an antibody fragment, or a small molecule drug. In some embodiments, the isolated GXM is isolated from C. neoformans. In some embodiments, the combination is formulated for intranasal administration. In some embodiments, the combination is formulated for intravascular administration.

Described are pharmaceutical combinations comprising a therapeutic molecule and an isolated GXM. The molecule can be, but is not limited to, a therapeutic molecule (i.e., a pharmaceutical agent or active pharmaceutical ingredient). The therapeutic molecule can be, but is not limited to, a protein, an antibody, an antibody fragment, or a small molecule drug. In some embodiments, the isolated GXM is isolated from C. neoformans. In some embodiments, the therapeutic molecule and the isolated GXM are formulated for administration to a subject separately. In some embodiments, the isolated GXM is formulated for intranasal or intravascular administration.

### BRIEF DESCRIPTION OF THE DRAWINGS

FIG. 1. Exogenous glucuronoxylomannan (GXM) administration and Cryptococcus neoformans H99 strain pulmonary infection reduced survival in C57BL/6 mice. (Panel A) Experimental timeline for the intranasal (i.n.) GXM challenge and fungal intratracheal infection model. Mice were sensitized with 125 μg/mL of GXM 24 h pre-inoculation with 10⁵ C. neoformans cells. Then, survival studies and colony forming units (CFU) determinations, histopathology, and microscopy were performed 3- and 7-days post-infection (dpi). (Panel B) Survival differences of untreated (e.g., sterile phosphate-buffered saline (PBS)-sensitization) and GXM-treated C57BL/6 mice infected with 10⁵ fungi (n=10 per group). P-value significance (*P<0.05) was calculated by log rank (Mantel-Cox) analysis. This experiment was performed twice, similar results were obtained each time, and all the results combined are presented.

FIG. 2. GXM exacerbates pulmonary cryptococcosis and systemic fungal load. Fungal burden in (Panel A) lungs (numbers of CFU/gram (g) of tissue) and (Panel B) blood (numbers of CFU/0.1 mL of blood) collected from untreated and GXM-treated mice i.t. infected with 10⁵ cryptococci (n=8 per group) 3- and 7-dpi. For panels B and C, bars and error bars denote the means and standard deviations (SDs), respectively. Asterisks indicate P-value significance (**P<0.01 and ****P<0.0001) calculated using multiple student's t-test analyses. ns represents not statistically significant comparisons. This experiment was performed twice, similar results were obtained each time, and all the results combined are presented. (Panel C) Histological analysis of lungs removed from untreated (left panels) and GXM-treated (right panels) C57BL/6 mice infected with 10⁵ C. neoformans H99 strain cells. Representative H&E (host tissue morphology; upper panels; scale bar: 100 μm)- and mucin carmine (fungi; red staining; lower panels)-stained sections of the lungs are shown. Tissues were removed 7-dpi. Lower panel images are magnifications of the smaller boxes in the H&E-stained sections to better show the cryptococcal cells stained with mucin carmine (indicated with arrows; scale bar: 20 μm).

FIG. 3. Exogenous GXM challenge enhanced C. neoformans migration into the central nervous system (CNS) of C57BL/6 mice. (Panel A) Fungal burden (numbers of CFU/gram of tissue) in brain collected from untreated and GXM-treated mice i.t. infected with 10⁵ cryptococci (n=8 per group) 3- and 7-dpi. Bars and error bars denote the means and SDs, respectively. Asterisks denote P-value significance (*P<0.05 and ****P<0.0001) calculated using multiple student's t-test analyses. (Panel B) Confocal microscopy of cryptococcal cells (arrowheads) in the brain parenchyma of GXM-treated mice. Scale bar: 20 μm. (Panel C) Immunofluorescent (IF) image of a hippocampal tissue section displaying a large cryptococcoma (arrows) filled with yeasts cells (black arrowhead) and abundant amounts of capsular polysaccharide released (white arrowheads). Scale bar: 150 μm. (Panel D) GXM accumulation (arrowheads) in the cortex and (Panel E) blood vessel of GXM-treated and C. neoformans infected mice. Scale bar: 200 μm. For panels B to E, FITC-labeled GXM-specific monoclonal antibody (mAb) 18B7 was used to label cryptococci. MAP-2 and DAPI staining were used to label the cell bodies and nuclei of neurons, respectively.

FIG. 4. Mice sensitized with exogenous GXM exhibited more and larger brain lesions or cryptococcomas than untreated animals. (Panel A) IF staining of brain lesions (cryptococcomas; arrows) caused by C. neoformans 7-dpi in untreated or GXM-treated animals. Fluorescein isothiocyanate (FITC)-labeled capsular-specific mAb 18B7 was used to label fungal cells. MAP-2 and DAPI staining were used to label the cell bodies and nuclei of neurons, respectively. Scale bar: 150 μm. (Panel B) Brain lesions per mouse (n=7 per group) and (Panel C) lesion area (n=10 per group) analyses in tissue sections of untreated and GXM-treated mice infected with C. neoformans. The areas of 10 brain lesions per condition were measured using NIH ImageJ software. Each symbol represents a single lesion. Bars and error bars denote the mean value and SDs, respectively. Asterisks denote P-value significance (*P<0.05 and ***P<0.001) calculated using student's t-test analysis.

FIG. 5. GXM challenge altered tight junction protein (TJ) expression in vivo. (Panel A) Confocal microscopy of cryptococcal GXM [FITC-labeled (arrowheads) GXM-specific mAb 18B7] distribution in the brain parenchyma (nuclei of neurons) of GXM-treated mice 24 h after i.n. administration. Scale bar: 20 μm. (Panel B) Western blot (WB) analyses of brain tissue from untreated (e.g., sterile PBS-sensitization) or GXM (7.8, 15.6, 31.25, 62.5, and 125 μg/mL)-challenged C57BL/6 mice were performed to compare the expression of the TJ proteins claudin-5 (23 kDa), ZO-1 (200 kDa), and occludin (59 kDa) and the adhesion protein JAM-A (32 kDa). Actin (42 kDa) was used as a housekeeping protein control. Quantitative measurements of individual band intensity in western blot analyses described in panel B for (Panel C) claudin-5, (Panel D) ZO-1, (Panel E) occludin, and (Panel F) JAM-A, using NIH ImageJ software. Actin was used as a control to determine the relative intensity ratio (A.U. denotes arbitrary units). Bars represent the means of 5 independent gel results (black circles) and error bars indicate SDs. Asterisks denote P-value significance calculated using ANOVA and adjusted using the Tukey's post-hoc analysis. *P<0.05, for the reduction in the intensity of the band of TJ and adhesion molecules as compared to actin.

FIG. 6. GXM altered the distribution of TJs on human brain endothelial cells (HBECs). (Panel A) IF images of claudin and occludin distribution on the surface of HBECs after incubation in absence (untreated) and presence of 10 μg/mL of ethylenediaminetetraacetic acid (EDTA; positive control) or GXM for 4 h at 37° C. and 5% CO₂. After co-incubation with EDTA or GXM, the cells were washed and incubated with claudin-FITC and occludin-rhodamine-specific antibodies. DAPI was used to stain the cell nuclei. Scale bar, 100 μm. (Panel B) Quantification of (Panel B) claudin and (Panel C) occludin intensity on the surface of HBECs was performed using NIH ImageJ software. Bars and error bars denote the means and SDs, respectively. Each symbol denotes a single cell measurement (n=10 cells per group). Asterisks indicate P-value significance (*P<0.05, **P<0.01 and ****P<0.0001) calculated using ANOVA and adjusted using the Tukey's post-hoc analysis. ns represents not statistically significant comparisons.

FIG. 7. GXM augmented the expression of activated RhoA in HBECs in a time-dependent manner. (Panel A) Western Blot analyses of HBEC lysates stimulated with 10 μg/mL of GXM were performed to assess the expression of activated RhoA protein (GTP-bound) over the course of 180 min. Actin was used as a protein control. Quantitative measurements of individual band intensity in western blot analyses described in panel A for (Panel B) activated RhoA using NIH ImageJ software. Bars represent the means of 3 independent gel results (black circles) and error bars indicate SDs. Asterisks denote P-value significance calculated using ANOVA and adjusted using the Tukey's post-hoc analysis. ***P<0.001 represents a significant increase in activated RhoA after 30 min compared to the other time points.

FIG. 8. GXM decreased the trans-endothelial electrical resistance (TEER) and increased the permeability of HBECs in a blood brain barrier (BBB) model. Graphic representation of the HBECs and pericytes BBB model used for the (Panel A) TEER measurements and (Panel C) HBEC barrier permeability assay. HBECs and pericytes were grown separately (HBECs, 0.4 μm pore insert; pericytes, bottom of a well) and co-incubated using a microtiter transwell system that permits chemotactic exchange through the supernatant. (Panel B) Relative TEER of HBECs incubated with GXM (10, 50, or 100 μg/mL) for 5 h at 37° C. and 5% CO₂. HBECs incubated alone or with EDTA (10 μg/mL) were used as negative and positive controls, respectively. Time points are the averages of the results for three different TEER measurements (n=3 wells per group per experiment), and error bars denote SDs. Symbols (*, #, ¢, and x) indicate P value significance (P<0.05) calculated using ANOVA and adjusted using the Tukey's post-hoc analysis. *, #, φ, and x indicate significantly higher TEER than in the 10 μg/mL of EDTA-, 10 μg/mL of GXM-, 50 μg/mL of GXM-, and 100 μg/mL of GXM-treated groups, respectively. (Panel D) Relative HBEC barrier permeability to streptavidin-horseradish peroxidase (HRP) after incubation with GXM (10, 50, or 100 μg/mL) for 30 min at 37° C. and 5% CO₂. HBECs incubated alone or with EDTA (10 μg/mL) were used as negative and positive controls, respectively. Bars and error bars denote the means and SDs, respectively. Each symbol denotes a single well measurement (n=3 wells per group). Asterisks indicate P-value significance (*P<0.05, **P<0.01 and ***P<0.001) calculated using ANOVA and adjusted using the Tukey's post-hoc analysis. ns represents not statistically significant comparisons. These experiments were performed thrice, similar results were obtained each time, and all the results combined are presented.

FIG. 9. GXM increased the transmigration of C. neoformans through HBECs. (Panel A) HBECs were cultured for 2 h with 10 μg/mL of GXM in a microtiter transwell system (5 μm pore insert). Then, 10⁵ cryptococci were added to the well and fungal transmigration through the HBECs was determined for 4 h using CFU. (Panel B) CFU determinations after fungal transmigration are shown. HBECs incubated alone or with EDTA (10 μg/mL) were used as negative and positive controls, respectively. Bars and error bars denote the means and SDs, respectively. Each symbol denotes CFU determinations from a single well (n=5 wells per group). Asterisks indicate P-value significance (*P<0.05, **P<0.01 and ****P<0.0001) calculated using ANOVA and adjusted using the Tukey's post-hoc analysis. ns represents not statistically significant comparisons.

FIG. 10. GXM caused vasodilation of mouse blood vessels after ex vivo treatment. (Panel A) Murine superior mesenteric arteries (SMA) incubated in absence (untreated) or presence of 25 μg/mL GXM were evaluated in response to phenylephrine (PE; test for vasoconstriction) or acetylcholine (ACh; test for endothelium-dependent vasodilation). Cumulative concentration-response curves are shown. Each point represents the average of 5 individual SMA measurements. Concentration-response curve was log-transformed, normalized to percent maximal response, and fitted using a nonlinear regression. P values of <0.05 were considered significant. (Panel B) pD2 for ACh curve. Each symbol (n=5, untreated; n=3, GXM-treated SMA rings) represents a single SMA ring. Bars and error bars denote the mean value and SDs, respectively. Asterisk denotes P-value significance (*P<0.05) calculated using student's t-test analysis.

FIG. 11. Model of GXM-mediated disruption of the BBB. GXM stimulates activation of RhoA, which activates Rho Activated Kinase (ROCK). ROCK inhibits the myosin light chain phosphatase (MLCP), leading to actin stress fiber formation through enhancement of MLC phosphorylation. Actin stress fibers contribute to the internalization and lysosomal degradation of claudin and occludin, disrupting TJs between brain microvascular endothelial cells.

FIG. 12. Graphs illustrating GXM induced VE-cadherin remodeling at the plasma membrane of human EA.hy926 ECs grown in confluent monolayers. Fluorescence intensity of (A) plasma membrane VE-cadherin and (B) phalloidin-549 at the cell periphery measured using line-scan analysis. (C) Actin stress fiber anisotropy measured using the FibrilTool macro in ImageJ software. Each symbol represents a single measurement (n=6 per time point). Bars and error bars denote means and SDs, respectively. Significance (****, P<0.0001) was calculated by one-way ANOVA and adjusted using Tukey's post hoc analysis. A.U. refers to arbitrary units.

### DESCRIPTION

### I. Definitions

Definitions of certain terms are provided below. Other terms are set forth throughout the specification. Meanings of these and other terms in particular instances throughout this specification will be clear to those skilled in the art from context.

The singular form “a,” “an,” and “the” include plural references unless the context clearly dictates otherwise. Thus, for example, reference to an “agent” includes a plurality of agents. The conjunction “or” is to be interpreted in the inclusive sense, i.e., as equivalent to “and/or,” unless the inclusive sense would be unreasonable in the context. The term “and/or” refers to and encompasses any and all possible combinations of one or more of the associated listed items, as well as the lack of combinations when interpreted in the alternative (“or”).

As used herein, the terms “about” and “approximately,” when used to modify an amount specified in a numeric value or range, indicates the numeric value as well as reasonable deviations from the value known to the skilled person in the art. In some embodiments, the term “about” means within the typical ranges of tolerances in the art. In some embodiments, the term “about” means within 1 or 2 standard deviations from the mean. In some embodiments, the term “about” means±10%. In some embodiments, the term “about” means±5%. When the term “about” is present before a series of numbers or a range, it is understood that “about” can modify each of the numbers in the series or range.

The use of “comprise,” “comprises,” “comprising,” “contain,” “contains,” “containing,” “include,” “includes,” and “including” are not intended to be limiting. Unless specifically noted, embodiments in the specification that recite “comprising” various components are also contemplated as “consisting of” or “consisting essentially of” the recited components. Embodiments in the specification that recite “consisting essentially of” various components are also contemplated as “consisting of”. “Consisting essentially of” means that additional component(s), composition(s), or method step(s) that do not materially change the basic and novel characteristics of the compositions and methods described herein may be included in those compositions or methods.

All ranges are to be interpreted as encompassing the endpoints in the absence of express exclusions, such as “not including the endpoints”; thus, for example, “within 10-15” includes the values 10 and 15. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.). When values are expressed as approximations, by use of the antecedent “about,” it will be understood that the particular value forms a further aspect. For example, if the value “about 10” is disclosed, then “10” is also disclosed.

“Purifying” or “isolating” GXM, to form isolated or purified GXM, refers to separating GXM from Cryptococcus neoformans or from one or more non-GXM components of Cryptococcus neoformans. In some embodiments, purified or isolated GXM is separated from any viable Cryptococcus neoformans. In some embodiments, capsular material is separated from Cryptococcus neoformans and GXM is further purified from the capsular material. In some embodiments, isolated GXM indicates that the GXM is essentially microbe free. In some embodiments, the isolated GXM comprises a cell-free preparation of GXM. In some embodiments, the isolated GXM is essentially pure. In some embodiments, the isolated GXM is at least 60%, at least 65%, at least 70%, at least 75%, at least 80%, at least 85%, at least 90%, or at least 95% pure.

“Endothelial cells” form a single cell layer that lines all blood vessels and regulates exchanges between the bloodstream and the surrounding tissues.

“Pericytes” is the term for vascular mural cells embedded within the vascular basement membrane of blood microvessels, where they make specific focal contacts with the endothelium. Pericytes are related to vascular smooth muscle cells (VSMC). Pericytes are found around blood capillaries, precapillary arterioles, postcapillary venules, and collecting venules.

Cryptococcus neoformans (C. neoformans) is an encapsulated yeast belonging to the class Tremellomycetes and an obligate aerobe that can live in both plants and animals. The C. neoformans polysaccharide capsule is composed of at least three components: glucuronoxylomannan (GXM), galactoxylomannan, and mannoprotein.

The terms “treat,” “treatment,” and the like, mean the methods or steps taken to provide relief from, or amelioration or alleviation of the number, severity, adverse effect, and/or frequency of one or more symptoms or pathological consequences of a disease, disorder, or condition in a subject. Treatment can be prophylactic in terms of preventing or partially preventing a disease, or a symptom or condition of the disease. Preventing includes providing prophylaxis with respect to the occurrence or recurrence of a disease in a subject that may be predisposed to the disease but has not yet been diagnosed with the disease. Preventing also includes providing prophylaxis with respect to the occurrence or recurrence of a symptom or pathological consequence of a disease in a subject that may be predisposed symptom or pathological consequence of the disease but has not yet been diagnosed with the symptom or pathological consequence the disease. Treatment can also be prophylactic in terms of delaying onset of a disease, or a symptom or condition of the disease. Delaying development of a disease or symptom or pathological consequence of the disease indicates deferring, hindering, slowing, retarding, stabilizing, suppressing, and/or postponing development of the disease or symptom or pathological consequence of the disease. The delay can be of varying lengths of time, depending on the history of the disease and/or individual being treated. Treating can include inhibiting the disease, disorder or condition, e.g., impeding its progress; and relieving the disease, disorder, or condition, e.g., causing regression of the disease, disorder and/or condition. Treatment can also mean prolonging survival as compared to expected survival in the absence of treatment. Treatment can be therapeutic in terms of a partial or complete cure of a disease, condition, symptom or adverse effect attributed to the disease, disorder, or condition. The term treatment can include: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease and/or its symptoms or conditions. Treating can refer to both therapeutic treatment alone, prophylactic treatment alone, or both therapeutic and prophylactic treatment. Those in need of treatment (subjects in need thereof) can include those previously diagnosed with a disease, disorder, or condition, or those identified as being at risk of developing a disease, disorder, or condition. Treating the disease, disorder, or condition can include ameliorating at least one symptom of the particular disease, disorder, or condition, even if the underlying pathophysiology is not affected.

A “pharmacologically effective amount,” “therapeutically effective amount,” or simply “effective amount” refers to that amount (dose) of a described isolated GXM, active pharmaceutical ingredient, or pharmaceutical composition to produce the intended physiological, pharmacological, therapeutic, or preventive result. An “effective amount” can also refer to the amount of, for example an excipient, in a pharmaceutical composition that is sufficient to achieve the desired property of the composition. An effective amount can be administered in one or more administrations, applications, or dosages. An “effective amount” of an agent can refer to an amount effective, at dosages and for periods of time necessary, to achieve a desired physiological or therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered.

A “dose,” “unit dose,” or “dosage” refers to physically discrete units suitable for use in a subject, each unit containing a predetermined quantity of isolated GXM, pharmaceutical agent and/or a pharmaceutical composition.

A “subject” refers to an animal that is the object of treatment, observation, or experiment. “Animal” includes cold- and warm-blooded vertebrates and invertebrates such as fish, shellfish, reptiles and, in particular, mammals. In some embodiments, the subject is a mammal. Mammal includes, but is not limited to, mice, rats, rabbits, guinea pigs, dogs, cats, sheep, goats, cows, horses, primates, such as monkeys, chimpanzees, and apes, and humans. In some embodiments, the subject is a human.

### II. Glucuronoxylomannan for Use in Central Nervous System Delivery

Described is the use of isolated Glucuronoxylomannan (GXM) to increase delivery of a molecule to the central nervous system (CNS) in a subject. In some embodiments, isolated GXM is administered to a subject to increase delivery of a molecule to the CNS in a subject. In some embodiments, the isolated GXM is administered to the subject intranasally. In some embodiments, the isolated GXM is administered to the subject intravascularly (e.g., intravenously).

GXM is the principal constituent of the Cryptococcus neoformans capsule. GXM is made up of the monosaccharides glucuronic acid, xylose, and mannose and can also contain O-acetyl groups. Isolated GXM indicates that the GXM is not associated with Cryptococcus neoformans. Isolated GXM can be purified from one or more components of C. neoformans, or synthetically produced. In some embodiments, isolated GXM indicates that the GXM is essentially microbe free. Purification of GXM from one or more components of C. neoformans can be done using methods available in the art for purifying or isolating capsular components (e.g., GXM) from encapsulated yeasts (e.g., C. neoformans). In some embodiments, isolated GXM indicates that the GXM is essentially microbe free. In some embodiments, the isolated GXM comprises a cell-free preparation of GXM. In some embodiments, the isolated GXM is essentially pure. In some embodiments, the isolated GXM is at least 60%, at least 65%, at least 70%, at least 75%, at least 80%, at least 85%, at least 90%, or at least 95% pure.

Also described is the use of isolated GXM to increase permeability of the Blood Brain Barrier (BBB) in a subject. Increasing permeability of the BBB can include, but is not limited to, altering the distribution and/or number of endothelial cell tight junctions in a blood vessel of the CNS, altering endothelial cell tight junction protein expression in a blood vessel of the CNS, reducing trans-endothelial electrical resistance of endothelial cells in a blood vessel of the CNS, increasing Ras homolog family member A (RhoA) expression in endothelial cells in a blood vessel of the CNS, increasing RhoA activation in endothelial cells in a blood vessel of the CNS, increasing myosin light chain (MLC) phosphorylation in endothelial cells in a blood vessel of the CNS, forming actin stress fibers in endothelial cells, remodeling the cytoskeleton in endothelial cells in a blood vessel of the CNS, increasing blood vessel relaxation in a blood vessel of the CNS, weakening brain endothelial cell/pericyte interactions in a blood vessel of the CNS, or combinations thereof. BBB permeability in the subject can be increased transiently. In some embodiments, isolated GXM is administered to a subject to increase BBB permeability in the subject. In some embodiments, isolated GXM is administered to a subject to transiently increase BBB permeability in the subject. The increase in BBB permeability can persist for about 5 minutes to about 5 hours or more. In some embodiments, the isolated GXM is administered to the subject intranasally. In some embodiments, the isolated GXM is administered to the subject intravascularly (e.g., intravenously).

Described are pharmaceutical combinations for delivering a molecule to the CNS of a subject comprising, the molecule and an effective dose of isolated GXM. The molecule can be administered to the subject prior to administering the isolated GXM, concomitantly with administering the isolated GXM, or subsequent to administering the isolated GXM. In some embodiments, the isolated GXM is administered to the subject within 24 hours, within 18 hours, within 12 hours, within 8 hours, within 4 hours, within 2 hours, within 1 hour, within 0.5 hour, within 15 minutes, within 10 minutes, or within 5 minutes of administration of the molecule. In some embodiments, the molecule is administered to the subject within 24 hours, within 18 hours, within 12 hours, within 8 hours, within 4 hours, within 2 hours, within 1 hour, within 0.5 hour, within 15 minutes, within 10 minutes, or within 5 minutes of administration of the isolated GXM. The molecule can be a pharmaceutical agent. The molecule can be, but is not limited to, protein, an antibody, an antibody fragment, a biologic agent, or a small molecule drug. The molecule can be formulated for administration to the subject by any route and/or means suitable for the particular molecule. Suitable administration routes for the molecule include, but are not limited to, intravascular (e.g., intravenous), intranasal, and subcutaneous. In some embodiments, the isolated GXM is formulated for administration to the subject by intranasal administration. In some embodiments, the isolated GXM is formulated for administration to the subject by intravascular (e.g., intravenous) administration.

In some embodiments, the isolated GXM is administered to a subject at a concentration of about 5 μg/mL to about 200 μg/mL or about 10 μg/mL to about 200 μg/mL. In some embodiments, the isolated GXM is administered to a subject at a concentration of about 5 μg/mL, about 10 μg/mL, about 15 μg/mL, about 20 μg/mL, about 25 μg/mL, about 30 μg/mL, about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, about 85 μg/mL, about 90 μg/mL, about 95 μg/mL, about 100 μg/mL, about 110 μg/mL, about 120 μg/mL, about 130 μg/mL, about 140 μg/mL, about 150 μg/mL, about 160 μg/mL, about 170 μg/mL, about 180 μg/mL, about 190 μg/mL, or about 200 μg/mL.

The isolated GXM can be provided in a pharmaceutically acceptable composition. The pharmaceutical composition includes a pharmaceutically effective amount of isolated GXM and optionally one or more pharmaceutically acceptable excipients. Pharmaceutically acceptable excipients (excipients) are substances other than the isolated GXM or an Active Pharmaceutical ingredient (API, therapeutic product that are intentionally included in the pharmaceutical composition. Excipients do not exert or are not intended to exert a therapeutic effect at the intended dosage. Excipients may act to (a) aid in processing of isolated GXM and/or the API during manufacture, (b) protect, support or enhance stability, bioavailability, or patient acceptability of the isolated GXM and/or the API, (c) assist in product identification, and/or (d) enhance any other attribute of the overall safety, effectiveness, or delivery of the isolated GXM and/or the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The carrier can be, but is not limited to, a solvent or dispersion medium containing, for example, water, saline, phosphate buffered saline, Ringer's solution, ethanol, polyol (for example, glycerol, propylene glycol, and liquid polyethylene glycol), and suitable mixtures thereof. A carrier may also contain adjuvants or additives such as preservatives, wetting agents, emulsifying agents and dispersing agents. A carrier may also contain isotonic agents, such as sugars, polyalcohols, sodium chloride, and the like.

The pharmaceutical compositions can contain other additional components commonly found in pharmaceutical compositions. Such additional components can include, but are not limited to: anti-pruritics, astringents, local anesthetics, or anti-inflammatory agents (e.g., antihistamine, diphenhydramine, etc.).

Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals and more particularly in humans.

In some embodiments, the pharmaceutical compositions further comprises one or more Active Pharmaceutical Ingredients (APIs). The API can be a pharmaceutical agent (molecule) for which delivery to the CNS is desired or which is expected to have or exert an effect in the CNS of a subject. In some embodiments, the pharmaceutical agent (e.g., API) is known to be efficacious in tissues other than the brain or central nervous system, but does not typically cross the blood brain barrier in sufficient amounts or at a sufficient rate to be efficacious in the brain or central nervous system. The pharmaceutical agent can be, but is not limited to, protein, an antibody, an antibody fragment, a biologic agent, or a small molecule drug. In some embodiments, a pharmaceutical composition comprising isolated GXM and one or more pharmaceutical agents is formulated for administration to the subject by intranasal administration. In some embodiments, a pharmaceutical composition comprising isolated GXM and one or more pharmaceutical agent is formulated for administration to the subject by intravascular (e.g., intravenous) administration.

Described are pharmaceutical compositions for delivering a molecule to the CNS of a subject comprising, the molecule and an effective amount of isolated GXM. The molecule can be a pharmaceutical agent. The molecule can be, but is not limited to, protein, an antibody, an antibody fragment, a biologic agent, or a small molecule drug. In some embodiments, the pharmaceutical composition is formulated for administration to the subject by intranasal administration. In some embodiments, the pharmaceutical composition is formulated for administration to the subject by intravascular (e.g., intravenous) administration.

The isolated GXM, a composition containing the isolated GXM, or a composition comprising the isolated GXM and an API can be provided in a sterile formulation. Typically, a pharmaceutical composition for use in in vivo administration is sterile. Sterilization can be accomplished according to methods known in the art, e.g., heat sterilization, steam sterilization, sterile filtration, or irradiation.

Dosages and desired drug concentration of isolated GXM and/or API of the invention may vary depending on the particular use envisioned. The determination of the appropriate dosage or route of administration is well within the skill of one in the art.

In some embodiments, the isolated GXM is formulated with or formulated for use in combination with a therapeutic agent (API) useful for treating a disease or condition of the CNS, a neurological disease, a cancer of the CNS, or an infection in the CNS.

In some embodiments, methods of increasing BBB permeability in a subject are described, the methods comprising administering to the subject isolated GXM. In some embodiments, the isolated GXM is administered to the subject intranasally. In some embodiments, the isolated GXM is administered to the subject intravascularly (e.g., intravenously). In some embodiments, BBB permeability is increased transiently.

In some embodiments, methods of altering the distribution and/or reducing the number of endothelial cell tight junctions in a blood vessel of the CNS in a subject are described, the methods comprising administering to the subject isolated GXM. In some embodiments, the isolated GXM is administered to the subject intranasally. In some embodiments, the isolated GXM is administered to the subject intravascularly (e.g., intravenously). In some embodiments, distribution of endothelial cell tight junctions is altered transiently. In some embodiments, the number of endothelial cell tight junctions is reduced transiently.

In some embodiments, methods altering endothelial cell tight junction protein expression in a blood vessel of the CNS in a subject are described, the methods comprising administering to the subject isolated GXM. In some embodiments, the isolated GXM is administered to the subject intranasally. In some embodiments, the isolated GXM is administered to the subject intravascularly (e.g., intravenously). In some embodiments, cell tight junction protein expression is altered transiently.

In some embodiments, methods of reducing trans-endothelial electrical resistance of endothelial cells in a blood vessel of the CNS in a subject are described, the methods comprising administering to the subject isolated GXM. In some embodiments, the isolated GXM is administered to the subject intranasally. In some embodiments, the isolated GXM is administered to the subject intravascularly (e.g., intravenously). In some embodiments, trans-endothelial electrical resistance is reduced transiently.

In some embodiments, methods of increasing Ras homolog family member A (RhoA) expression in endothelial cells in a blood vessel of the CNS in a subject are described, the methods comprising administering to the subject isolated GXM. In some embodiments, the isolated GXM is administered to the subject intranasally. In some embodiments, the isolated GXM is administered to the subject intravascularly (e.g., intravenously). In some embodiments, RhoA expression is increased transiently.

In some embodiments, methods of increasing RhoA activation in endothelial cells in a blood vessel of the CNS in a subject are described, the methods comprising administering to the subject isolated GXM. In some embodiments, the isolated GXM is administered to the subject intranasally. In some embodiments, the isolated GXM is administered to the subject intravascularly (e.g., intravenously). In some embodiments, RhoA activation is increased transiently.

In some embodiments, methods of increasing myosin light chain (MLC) phosphorylation in endothelial cells in a blood vessel of the CNS in a subject are described, the methods comprising administering to the subject isolated GXM. In some embodiments, the isolated GXM is administered to the subject intranasally. In some embodiments, the isolated GXM is administered to the subject intravascularly (e.g., intravenously). In some embodiments, MLC phosphorylation is increased transiently.

In some embodiments, methods of forming actin stress fibers in endothelial cells in a blood vessel of the CNS in a subject are described, the methods comprising administering to the subject isolated GXM. In some embodiments, the isolated GXM is administered to the subject intranasally. In some embodiments, the isolated GXM is administered to the subject intravascularly (e.g., intravenously). In some embodiments, the actin stress fibers are formed transiently.

In some embodiments, methods of remodeling the cytoskeleton in endothelial cells in a blood vessel of the CNS in a subject are described, the methods comprising administering to the subject isolated GXM. In some embodiments, the isolated GXM is administered to the subject intranasally. In some embodiments, the isolated GXM is administered to the subject intravascularly (e.g., intravenously). In some embodiments, the cytoskeleton is remodeled transiently.

In some embodiments, methods of increasing blood vessel relaxation in a blood vessel of the CNS in a subject are described, the methods comprising administering to the subject isolated GXM. In some embodiments, the isolated GXM is administered to the subject intranasally. In some embodiments, the isolated GXM is administered to the subject intravascularly (e.g., intravenously). In some embodiments, the blood vessels are relaxed transiently.

In some embodiments, methods of weakening brain endothelial cell/pericyte interactions in a blood vessel of the CNS in a subject are described, the methods comprising administering to the subject isolated GXM. In some embodiments, the isolated GXM is administered to the subject intranasally. In some embodiments, the isolated GXM is administered to the subject intravascularly (e.g., intravenously). In some embodiments, the endothelial cell/pericyte interactions are weakened transiently.

In some embodiments, administering isolated GXM to a subject alters distribution of endothelial cells tight junctions. The endothelial cells can be in a blood vessel of the brain. Altering distribution of tight junctions on the surface of endothelial cells comprises decreasing the number of tight junctions on the surface of the endothelial cells. Decreasing the number of tight junctions on the surface of the endothelial cells indicates that the number of tight junctions on the surface of endothelial cells in a subject treated with isolated GXM is less than the number of tight junctions on the surface of endothelial cells in the subject prior to administering the isolated GXM to the subject or less than the number of tight junctions on the surface of endothelial cells in a control subject that has not been treated with isolated GXM. The number of tight junctions on the surface of endothelial cells in a subject treated with isolated GXM can be reduced by at least 10%, at least 20%, at least 30%, at least 40%, at least 50%, at least 60%, at least 70%, or at least 80% relative to the number of tight junctions on the surface of endothelial cells in the subject prior to administering the isolated GXM to the subject or to the number of tight junctions on the surface of endothelial cells in a control subject that has not been treated with isolated GXM.

In some embodiments, administering isolated GXM to a subject alters endothelial cell tight junction protein expression. The endothelial cells can be in a blood vessel of the brain. In some embodiments, altering endothelial cell tight junction protein expression comprises decreasing expression of claudin-5 and/or occludin in an endothelial cell. Decreasing the claudin-5 and/or occludin in endothelial cells indicates that the level of expression of claudin-5 and/or occludin in endothelial cells in a subject treated with isolated GXM is less than the level of expression of claudin-5 and/or occludin in endothelial cells in the subject prior to administering the isolated GXM to the subject or less than the level of expression of claudin-5 and/or occludin in endothelial cells in a control subject that has not been treated with isolated GXM. The level of expression of claudin-5 and/or occludin in endothelial cells in a subject treated with isolated GXM can be reduced by at least 10%, at least 20%, at least 30%, at least 40%, at least 50%, at least 60%, at least 70%, or at least 80% relative to the level of expression of claudin-5 and/or occludin in endothelial cells in the subject prior to administering the isolated GXM to the subject or to the level of expression of claudin-5 and/or occludin in endothelial cells in a control subject that has not been treated with isolated GXM.

In some embodiments, administering isolated GXM to a subject reduces trans-endothelial electrical resistance of endothelial cells. The endothelial cells can be in a blood vessel of the brain. Transendothelial electrical resistance (TEER) is the measurement of electrical resistance across a cellular monolayer and is a sensitive and reliable method to confirm the integrity and permeability of the monolayer. Reducing trans-endothelial electrical resistance in endothelial cells indicates that the level of trans-endothelial electrical resistance in an endothelial cell layer in a subject treated with isolated GXM is less than the trans-endothelial electrical resistance in an endothelial cell layer in the subject prior to administering the isolated GXM to the subject or less than the trans-endothelial electrical resistance in an endothelial cell layer in a control subject that has not been treated with isolated GXM. The trans-endothelial electrical resistance in an endothelial cell layer in a subject treated with isolated GXM can be reduced by at least 10%, at least 20%, at least 30%, at least 40%, at least 50%, at least 60%, at least 70%, or at least 80% relative to the trans-endothelial electrical resistance in an endothelial cell layer in the subject prior to administering the isolated GXM to the subject or to the trans-endothelial electrical resistance in an endothelial cell layer in a control subject that has not been treated with isolated GXM.

In some embodiments, administering isolated GXM to a subject increases Ras homolog family member A (RhoA) expression in endothelial cells. The endothelial cells can be in a blood vessel of the brain. Increasing RhoA expression in endothelial cells indicates that the level of expression of RhoA in endothelial cells in a subject treated with isolated GXM is more than the level of expression of RhoA in endothelial cells in the subject prior to administering the isolated GXM to the subject or more than the level of expression of RhoA in endothelial cells in a control subject that has not been treated with isolated GXM. The level of expression of RhoA in endothelial cells in a subject treated with isolated GXM can be increased by at least 10%, at least 20%, at least 30%, at least 40%, at least 50%, at least 60%, at least 70%, at least 80%, at least 90%, at least 100% relative to the level of expression of RhoA in endothelial cells in the subject prior to administering the isolated GXM to the subject or to the level of expression of RhoA in endothelial cells in a control subject that has not been treated with isolated GXM.

In some embodiments, administering isolated GXM to a subject increases RhoA activation in endothelial cells. The endothelial cells can be in a blood vessel of the brain. Increasing RhoA activation in endothelial cells indicates that the level of activation of RhoA in endothelial cells in a subject treated with isolated GXM is higher than the level of activation of RhoA in endothelial cells in the subject prior to administering the isolated GXM to the subject or higher than the level of activation of RhoA in endothelial cells in a control subject that has not been treated with isolated GXM. The level of activation of RhoA in endothelial cells in a subject treated with isolated GXM can be increased by at least 10%, at least 20%, at least 30%, at least 40%, at least 50%, at least 60%, at least 70%, at least 80%, at least 90%, at least 100% relative to the level of activation of RhoA in endothelial cells in the subject prior to administering the isolated GXM to the subject or to the level of activation of RhoA in endothelial cells in a control subject that has not been treated with isolated GXM.

In some embodiments, administering isolated GXM to a subject increases myosin light chain (MLC) phosphorylation in endothelial cells. The endothelial cells can be in a blood vessel of the brain. Increasing MLC phosphorylation in endothelial cells indicates that the level of MLC phosphorylation in endothelial cells in a subject treated with isolated GXM is more than the level of MLC phosphorylation in endothelial cells in the subject prior to administering the isolated GXM to the subject or more than the level of MLC phosphorylation in endothelial cells in a control subject that has not been treated with isolated GXM. The level of MLC phosphorylation in endothelial cells in a subject treated with isolated GXM can be increased by at least 10%, at least 20%, at least 30%, at least 40%, at least 50%, at least 60%, at least 70%, at least 80%, at least 90%, at least 100% relative to the level of MLC phosphorylation in endothelial cells in the subject prior to administering the isolated GXM to the subject or to the level of MLC phosphorylation in endothelial cells in a control subject that has not been treated with isolated GXM.

In some embodiments, administering isolated GXM to a subject results in formation of actin stress fibers in endothelial cells. The endothelial cells can be in a blood vessel of the brain. Actin Stress fibers are contractile bundles containing actin and found in non-muscle cells. Formation of actin stress fibers in endothelial cells indicates that the level of actin stress fibers in endothelial cells in a subject treated with isolated GXM is higher than the level of actin stress fibers in endothelial cells in the subject prior to administering the isolated GXM to the subject or more than the level of actin stress fibers in endothelial cells in a control subject that has not been treated with isolated GXM. The level of actin stress fibers in endothelial cells in a subject treated with isolated GXM can be increased by at least 10%, at least 20%, at least 30%, at least 40%, at least 50%, at least 60%, at least 70%, at least 80%, at least 90%, at least 100% relative to the level of actin stress fibers in endothelial cells in the subject prior to administering the isolated GXM to the subject or to the level of actin stress fibers in endothelial cells in a control subject that has not been treated with isolated GXM.

In some embodiments, administering isolated GXM to a subject results in remodeling the cytoskeleton in endothelial cells. The endothelial cells can be in a blood vessel of the brain. Remodeling the cytoskeleton in endothelial cells includes, but is not limited to, translocation of actin from the periphery of an endothelial cell to a perinuclear localization. Remodeling the cytoskeleton in endothelial cells indicates that the localization of cytoskeletal elements is altered in endothelial cells in a subject treated with isolated GXM relative to the localization of the cytoskeletal elements in endothelial cells in the subject prior to administering the isolated GXM to the subject or to the localization of the cytoskeletal elements in endothelial cells in a control subject that has not been treated with isolated GXM.

In some embodiments, administering isolated GXM to a subject increases blood vessel relaxation. The blood vessel can be a blood vessel of the brain. In some embodiments, increasing blood vessel relaxation comprises widening of blood vessels (i.e., vasodilation). Increasing blood vessel relaxation indicates that the level of blood vessel relaxation in a subject treated with isolated GXM is more than the level of blood vessel relaxation in endothelial cells in the subject prior to administering the isolated GXM to the subject or more than the level of blood vessel relaxation in endothelial cells in a control subject that has not been treated with isolated GXM. The level of blood vessel relaxation in endothelial cells in a subject treated with isolated GXM can be increased by at least 10%, at least 20%, at least 30%, at least 40%, at least 50%, at least 60%, at least 70%, at least 80%, at least 90%, at least 100% relative to the level of blood vessel relaxation in endothelial cells in the subject prior to administering the isolated GXM to the subject or to the level of blood vessel relaxation in endothelial cells in a control subject that has not been treated with isolated GXM.

In some embodiments, administering isolated GXM to a subject weakens brain endothelial cell/pericyte interactions. Weakening brain endothelial cell/pericyte interactions includes, but is not limited to, modulating communication (e.g., signaling) between pericytes and endothelial cells and decreasing adherence or contact between pericytes and endothelial cells.

Weakening brain endothelial cell/pericyte interactions indicates that the endothelial cell/pericyte interactions in a subject treated with isolated GXM are decreased relative to the endothelial cell/pericyte interactions in the subject prior to administering the isolated GXM to the subject or relative to the endothelial cell/pericyte interactions in a control subject that has not been treated with isolated GXM.

### III. Methods of Use

The described methods can be used to increase delivery of a molecule, such as a therapeutic agent (e.g., API) to the CNS of a subject. The methods can be used to increase the concentration of the pharmaceutical agent in the CNS and/or to increase the effectiveness of a pharmaceutical agent whose target is in the CNS. In some embodiments, the pharmaceutical agent is known to be efficacious in tissues other than the brain or central nervous system, but does not typically cross the blood brain barrier in sufficient amounts or at a sufficient rate to be efficacious in the brain or central nervous system. In some embodiments, the pharmaceutical agent is known to be efficacious in the brain or central nervous system, but whose effectiveness would be improved by increasing delivery to the brain or central nervous system.

The isolated GXM and methods of using the isolated GXM can be used in the treatment of a subject having a neurological disease or condition, a tumor (e.g., cancer) of the CNS, or an infection of the CNS. While not having a direct effect on the neurological disease or condition, the tumor, or the infection, administering isolated GXM to the subject can improve the efficacy of a pharmaceutical agent in treating the neurological disease or condition, the tumor, or the infection by increasing delivery of the pharmaceutical agent to the brain or CNS.

A brain tumor or cancer can be, but is not limited to, a glioma, an astrocytoma, a glioblastoma, an oligodendroglioma, an ependymoma, a choroid plexus tumors, a choroid plexus carcinoma, an embryonal tumor, a medulloblastoma, a germ cell tumor, a pineal tumor, a pineoblastoma, a meningioma, a nerve tumor, an acoustic neuroma, a pituitary tumor, a craniopharyngioma, a pituitary adenoma, a neuroblastoma, an olfactory neuroblastoma, chondrosarcoma, or a metastatic cancer that originated in another area of the body (e.g., a metastatic breast, colon, kidney lung, or skin (melanoma) cancer).

A neurological disease or disorder can be, but is not limited to, Alzheimer's disease, dementia, epilepsy, a seizure disorder, Parkinson's disease, a movement disorder, amyotrophic lateral sclerosis, cerebral palsy, chronic traumatic encephalopathy, Lewy body dementia, Huntington's disease, Mal de débarquement syndrome, migraine, and Tourette syndrome.

A CNS infection or infection-related diseases can be, but are not limited to, a bacterial infection, a viral infection, a parasitic infection, encephalitis, meningitis, bacterial meningitis, viral meningitis, progressive multifocal leukoencephalopathy, cysticercosis, schistosomiasis, toxoplasmosis, or Naegleria fowleri infection.

The isolated GXM and the pharmaceutical agent can be administered to the subject together or separately. For separate administration, the pharmaceutical agent can be administered to the subject prior to, concomitantly with, or subsequently to administration of the isolated GXM to the subject. In some embodiments, the isolated GXM and the pharmaceutical agent are administered to the subject intranasally. In some embodiments, the isolated GXM and the pharmaceutical agent are administered to the subject intravascularly (e.g., intravenously). In some embodiments, the isolated GXM is administered intranasally and the pharmaceutical agent is administered intranasally or by another administration route (e.g., intravascularly or subcutaneously).

Although the present invention has been described in some detail by way of illustration and example for purposes of clarity and understanding, it will be apparent that certain changes and modifications may be practiced within the scope of the appended claims.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Examples 1. Methods

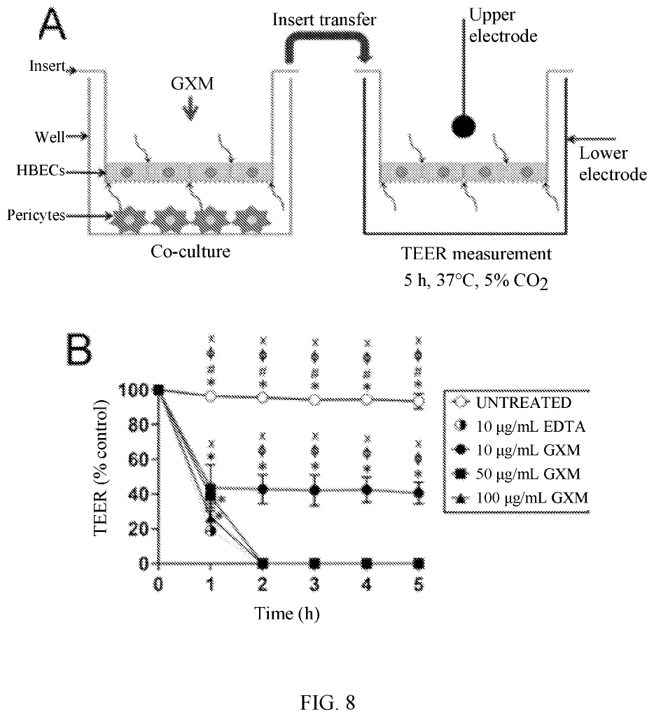

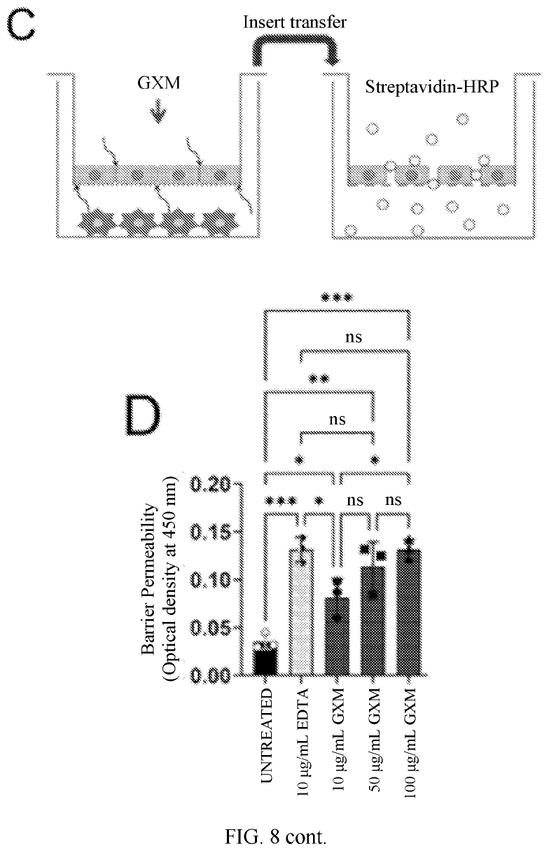

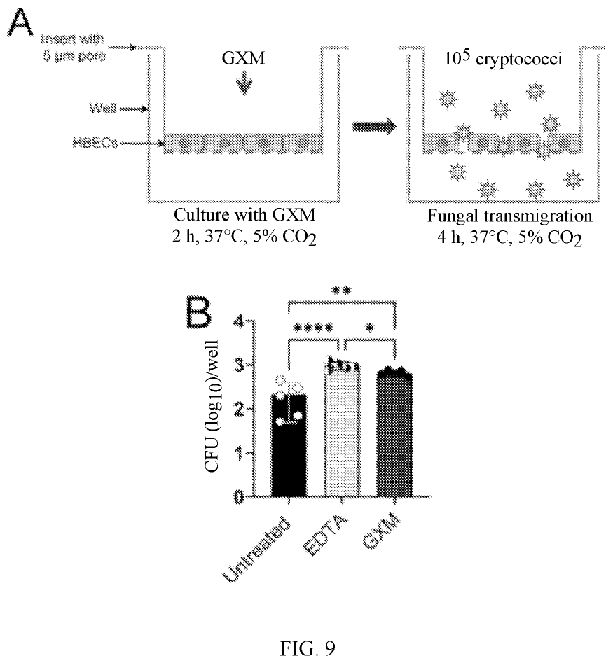

A. C. neoformans strain H99 (serotype A) was grown in Sabouraud dextrose broth (pH 5.6; BD Difco) for 24 h at 30° C. in an orbital shaker set at 150 rpm to early stationary phase.
B. In vivo GXM sensitization and pulmonary model of C. neoformans infection. Male and female C57BL/6 mice (10-12 weeks old; Charles Rivers) were anesthetized using the ketamine (100 mg/kg)-xylazine (10 mg/kg) cocktail and intranasal challenged with a 50 μL solution of phosphate-buffered saline (PBS; untreated) or GXM (125 μg/mL) in PBS 24 h prior to cryptococcal pulmonary infection. Then, animals were anesthetized using the ketamine-xylazine cocktail and a vertical 5-mm incision was made in the skin of the ventral neck just right of midline. The trachea was identified and injected with a 100 μL suspension containing 10⁵ C. neoformans strain H99 yeast cells using a 26-gauge syringe. The anterior neck site was closed with surgical glue and 1% topical chlorhexidine solution was applied over the closed incision. A group of mice was monitored for survival studies. A separate group of GXM challenged/infected mice were bled from the facial vein (0.1 mL blood collected), euthanized in a chamber with 30-70% CO₂ flow, and brain/lungs excised for CFU count determinations and histological processing.
C. Determination of GXM levels. C. neoformans capsular GXM in organs and serum were measured by capture ELISA. Organ tissue (0.2 g) was placed in 2 mL of PBS, homogenized, and the supernatant stored at −20° C. until quantified. Briefly, microtiter polystyrene plates were coated with goat anti-mouse (GAM) IgM (1 μg/mL) and blocked with 1% BAS in PBS. Next, the IgM GXM binding mAb 2D10 (2 μg/mL) was added as the capture antibody and the plate was incubated for 1 h. Culture supernatants were serially diluted on the plate and incubated for 1 h. The ELISA was completed by adding, in successive steps, mAb 18B7 (2 μg/mL) in buffer (PBS with 1% BSA), 1 μg of alkaline phosphatase-labeled GAM IgG1/mL in buffer, and 50 μL of p-nitrophenyl phosphate (5 mg/mL) in substrate buffer (0.001 M MgCl₂ and 0.05 M Na₂CO₃, in 1 L (pH 9.8)). Between each step, the wells were washed with 0.05% Tween 20 in tris-buffered saline (TBST). All incubations were done at 37° C. for 1 h or 4° C. overnight.
D. CFU determinations. Lung and brain tissues were excised from euthanized mice 3- and 7-dpi. The right lobe of lung and the right hemisphere of the brain were homogenized in 5 mL of sterile PBS, serially diluted, a 100 μL suspension was plated on Sabouraud dextrose agar (BD Difco) and incubated at 30° C. for 48 h. Quantification of viable yeast cells from untreated and GXM-challenged animals were determined by CFU counting.
E. Histological examinations.
(i) Lungs. Lung tissue was excised from euthanized mice 7-dpi and fixed in 10% formalin for 24 h, processed, and embedded in paraffin. Four-micrometer vertical sections were cut and then fixed to glass slides and subjected either to hematoxylin and eosin (H&E) stain or to mucin carmine stain, to examine host tissue and fungal morphology, respectively. Microscopic examinations of tissues were performed sequentially by light microscopy with a Leica DMi8 inverted microscope (20× and 40× objectives), and photographed with a Leica DFC7000 T digital camera using the Leica software platform LAS X. Multiple slides were visualized and examined to understand host tissue inflammation and fungal distribution.
(ii) Brain. C. neoformans cells and capsular polysaccharide released in tissue were stained using GXM-specific monoclonal antibody (mAb) 18B7. Slides were blocked, and mAb 18B7 (2 μg/ml) was added for 1 h at 37° C. After the slides were washed, fluorescein isothiocyanate (FITC)-conjugated GAM Ab (1:250 dilution; 1% bovine serum albumin (BSA)) was applied for 1 h at RT. Neurons in tissue section were stained with DAPI (4′,6-diamidino-2-phenylindole) and microtubule-associated protein-2 (MAP-2). Microscopic examinations of brain sections were performed with a fully motorized Carl Zeiss Axio Observer Z1 confocal microscope. Confocal images of blue, green, and red fluorescence were conceived simultaneously using a multichannel mode. Z-stack images and measurements were corrected utilizing Zen software in deconvolution mode.
F. Western blot analysis of mouse brain cells. Western blot analysis was conducted using cytoplasmic extracts from mouse brain cells made with a NE-PER nuclear and cytoplasmic extraction kit. The mixture was centrifuged at 10,000×g for 10 min at 4° C., and the resulting protein content of the supernatant was determined using the Bradford method, employing a Pierce BCA protein assay kit. Lysates were preserved in a Protease inhibitor cocktail and stored at −20° C. until use. Extracts were diluted with 2× Laemmli sample buffer and β-mercaptoethanol. The mixture was heated to 90° C. for 5 min. 23 μg of protein were applied to each lane of a gradient gel (7.5%). Proteins were separated by electrophoresis at a constant 130 V/gel for 90 min and transferred to a nitrocellulose membrane on the Trans-Blot Turbo Transfer System (Bio-Rad) at 25 V for 7 min. The membranes were blocked with 5% BSA in tris-buffered saline (TBST; 0.1% Tween 20) for 2 h at RT. A primary claudin-5-, ZO-1-, occludin-, or JAM-A-specific mAb (dilutions, 1:1000; Santa Cruz Biotechnology) was incubated overnight at 4° C. with TBST (5% BSA). After washing the membranes 3× with TBST for 10 min, a GAM IgG (H+L) conjugated to horseradish peroxidase was used as a secondary Ab (dilution, 1:1000; Southern Biotech) and incubated with TBST (5% BSA) for 1 h at RT. The membranes were washed as described above. Protein bands were measured using the UVP ChemStudio imaging system (Analytik Jena) after staining each membrane with chemiluminescence detection reagents (TF). Quantitative measurements of individual band intensities in western blot analyses for claudin-5, ZO-1, occludin, or JAM-A were performed using the NIH ImageJ software. Actin (dilution, 1:1,000; BD Biosciences), a cytoskeleton housekeeping protein, was used as loading controls to determine the relative intensity ratio.
G. Western blot analysis of HBEC signaling assay. Whole cell lysate was collected from Human Brain Endothelial Cells-5i (HBECs, American Type Culture Collection [ATCC]) grown in 24-well tissue culture dishes in 2× Laemmli Sample Buffer. Samples were incubated for 5 min at 95° C. to denature proteins and then loaded onto 4%-20% gradient SDS-PAGE gels. Proteins were transferred to nitrocellulose blotted with RhoA-GTP, RhoA (Pan) ROCK, phosphor-MLC, and MLC mAbs diluted (1:1000) in 1×TBST (1% BSA). Western blots were then incubated with GAM 680 and GA-rabbit 800 (LiCOR Biosciences) secondary Abs. Revert 520 Total protein stain (LiCOR) was used for western blot normalization. Blot images were collected using an Odyssey FC imager (LiCOR) and protein levels were quantified using LiCOR Image Studio software.
H. IF confocal microscopy. HBECs were seeded at 0.75×10⁵ cells/glass coverslips in 24-well tissue culture plates and cultured for 24 h at 37° C. and 5% CO₂. Then, monolayers of cells were incubated without or with 10 μg/mL of GXM for 4 h at 37° C. and 5% CO₂. EDTA (10 μg/mL) was used as a positive control. After each incubation, the solution with either GXM or EDTA was removed, and each coverslip fixed in a 4% paraformaldehyde solution for 10 min. Then, HBECs were rinsed 3× with PBS and blocked with PBS supplemented with 1% BSA followed by the addition of claudin-5-conjugated FITC and occludin-conjugated rhodamine binding mAbs (dilutions, 1:1000) in 1% BSA. Actin was stained with alexa-fluor phalloidin-594 (dilution, 1:500). The plate was then incubated for 1 h at 37° C. DAPI was diluted in PBS and used to stain HBEC nuclei for 1 h at RT. Slides were examined by confocal microscopy as described above and analyzed using NIH ImageJ software. Individual cells in each IF image were traced, the intensity of each protein determined, and the mean intensity per condition reported.
I. BBB model. HBECs were seeded at 0.75×10⁵ cells/well in 0.4 μM pore transwells (Greiner Bio-ONE) and grown at 37° C. Concurrently, human primary pericytes (Sciencell) were seeded at 1.5×10⁵ cells/well in 24-well tissue culture plates. After 24 h, transwells containing HBECs were transferred to wells containing pericytes and grown for 48 h.
(i) Trans-Endothelial Electrical Resistance. Relative trans-endothelial electrical resistance (TEER) of HBECs incubated with GXM (10, 50, 100 μg/mL) for 5 h at 37° C. and 5% CO₂ was recorded. HBECs incubated alone or with EDTA (10 μg/mL) were used as negative and positive controls. Respectively. TEER was performed for 5 h using a CellZScope-E (nano Analytics). FIG. 8 panel A illustrates the HBECs and pericytes BBB model used for the TEER measurements.
(ii) Streptavidin-HRP HBEC Barrier Permeability. Cells were stimulated with GXM for 1 h. Permeability of barriers was assayed by adding streptavidin-HRP (BD Biosciences) to the upper chamber and collecting samples from the bottom chamber after 30 min incubation at 37° C. and 5% CO₂. Samples were treated with 3,3′,5,5′ tetramethylbenzidine and quenched with 2 M H₂PO₄ in a 96-well plate. Absorbance was measured at 450 nm. FIG. 8 Panel B depicts the BBB model used for the endothelial cell barrier permeability assays.
J. Fungal transmigration studies. The in vitro BBB model consists of primary HBECs seeded at a density of 10⁵ in 5 μm pore transwells and grown for 24 h at 37° C., 5% CO₂. Then, we analyzed the passage of C. neoformans (10⁵ cryptococci) through the BBB in vitro for 4 h to determine the impact of GXM (10 μg/mL) on the integrity of the barrier. Untreated or EDTA (10 μg/mL)-treated tissue constructs were used as controls. CFU determinations after fungal transmigration were performed. FIG. 9 Panel A shows the HBECs BBB model used for the cryptococcal transmigration determination.
K. Vascular reactivity to GXM. Isolated superior mesenteric arteries (SMA) were incubated in oxygenated Krebs buffer (130 mM NaCl, 14.9 mM NaHCO₃, 4.7 mM KCl, 1.18 mM KH₂PO₄, 1.17 mM MgSO₄·7H₂O, 1.56 mM CaCl₂·2H₂O, 0.026 mM EDTA, 5.5 mM glucose, pH 7.4), with the perivascular fat carefully removed. SMA were cut into rings (2 mm in length) and cultured in vascular medium (ATCC) in an incubator at 37° C. supplied by 5% CO₂. SMA were treated ex vivo with 25 μg/mL of GXM for 40 min. The concentration of GXM was chosen based on results from TEER and HBEC barrier permeability experiments. A stock solution of GXM was made by dissolving the polysaccharide in distilled water and further diluted in Krebs solution. A vehicle consisting of distilled water was used as a control (untreated). After treatment, SMA rings were mounted in a Multi-Wire Myograph System 620M (Danish Myo Technology) for isometric tension recordings using a PowerLab 8/35 data acquisition system (ADInstruments Pty Ltd.). SMA rings were equilibrated in Krebs buffer for 30 min, and in chambers perfused with 5% CO₂ in 95% O₂ at 37° C. In all experiments, SMA ring integrity was assessed by stimulation with 120 mM KCl (74.7 mM NaCl, 14.9 mM NaHCO₃, 60 mM KCl, 1.18 mM, KH₂PO₄, 1.17 mM MgSO₄·7H₂O, 1.6 mM CaCl₂·2H₂O, 0.026 mM EDTA, 5.5 mM glucose). To test for the presence of endothelium, segments were contracted with 1 μM phelynephrine (PE); once the vessels reached a stable maximum tension, the vessels were stimulated with 10 μM acetylcholine (ACh), and relaxation was confirmed. For the stock solution, PE and ACh were dissolved in distilled water. SMA that achieved relaxation to ACh were considered to have a preserved endothelium. Cumulative concentration response curves to ACh (1 nM to 10 μM) and PE (1 nM to 10 μM) were performed on intact SMA rings in the absence or presence of 25 μg/mL of GXM. Endothelium-dependent relaxation was recorded for ACh after maximal precontraction with 1 μM PE.
L. Statistical analysis. All data were subjected to statistical analysis using Prism 9.4 (GraphPad). Differences in survival rates were analyzed by the log-rank test (Mantel-Cox). P values for multiple comparisons were calculated by one-way analysis of variance (ANOVA) and were adjusted by use of Tukey's post-hoc analysis. P values for individual comparisons were calculated using student's or multiple t-test analyses. Concentration-response curve for vascular reactivity was log-transformed, normalized to percent maximal response, and fitted using a nonlinear regression. P values of <0.05 were considered significant.

### Example 2. GXM Isolation

GXM was isolated from C. neoformans strain H99 using the hexadecyltrimethyl ammonium bromide (CTAB) method as described Nimrichter L et al. 2007 (Self-aggregation of Cryptococcus neoformans capsular glucuronoxylomannan is dependent on divalent cations. Eukaryot Cell. 2007; 6(8): 1400-10) with a few modifications. Briefly, fungal cells (10⁹) were inoculated into 1,000-mL Erlenmeyer flasks containing 400 mL of minimal medium composed of glucose (15 mM), MgSO₄ (10 mM), KH₂PO₄ (29.4 mM), glycine (13 mM), and thiamine-HCL (3 μM), pH 5.5. Fungal cells were cultivated for 3 days at 30° C. with shaking and separated from culture supernatants by centrifugation at 4,000×g (15 min, 4° C.). The supernatant fluids were collected and centrifuged again at 15,000×g (15 min, 4° C.) to remove smaller debris. The pellets were discarded, and the resulting supernatant was concentrated approximately 20-fold using an Amicon (Millipore) ultrafiltration cell (with a cutoff of 100 kDa and a total capacity of 200 mL) with stirring and Biomax polyethersulfone ultrafiltration discs (63.5 mm). A nitrogen (N₂) stream was used as the pressure gas. After the supernatant was concentrated, a thick, translucent film was observed in close association with the ultrafiltration disc and was covered by a concentrated fluid phase. The fluid phase was discarded, and the viscous layer was collected with a cell scraper for storage at room temperature (RT). Fractions that were passed through the 100-kDa filtration discs were filtered through 10-kDa membranes, resulting again in film formation. We heat inactivated (100° C. for 15 min) proteases in our GXM preparation. Additionally, each preparation was treated with protease inhibitor cocktail (37° C. for 2 h). Each preparation was also tested for contamination with bacterial lipopolysaccharide (LPS) using the Limulus amoebocyte lysate (LAL) assay (Lonza). LPS was undetected in the LAL assay. We performed CFU determinations in our preparations and did not observe any culturable microbial growth. For polysaccharide quantification, a capture ELISA, the carbazole reaction for hexuronic acid, and the method for hexose detection were used.

### Example 3. GXM Concentrations in Murine Tissues 24 h Post-Administration

To explore the impact of exogenous GXM on C. neoformans H99 strain infection, we first determined whether GXM disseminated systemically from the airways and could be detected in serum and brain tissue (Table 1). C57BL/6 mice (n=5) were challenged intranasally with a single GXM dose of 125 μg/mL, and GXM levels were quantified by enzyme-linked immunosorbent assay (ELISA) 24 h later. Lung tissue GXM levels were 2.17±0.09 μg/g.

GXM concentrations in serum and brain tissue were 0.34±0.05 μg/mL and 0.21±0.13 μg/g, respectively. As a control experiment, GXM levels were measured in lungs, serum, and brain collected from mice only infected with the fungus intratracheally (no GXM challenge) 3- and 7-days post-infection. The levels of GXM in pulmonary tissue 3- and 7-days post-infection were the highest with 13.6 μg/g (range: 10 to 18 μg/g) and 17.9 μg/g (range: 13 to 22 μg/g), respectively. Brain tissue evinced GXM concentrations of 4.4 μg/g (range: 2 to 7 μg/g, 3-dpi) and 12.8 μg/g (range: 9 to 19 μg/g, 7-dpi). Additionally, circulating GXM in serum was 4.75 μg/mL (range: 2 to 7 μg/mL, 3-dpi) and 10.25 μg/mL (range: 7 to 14 μg/mL, 7-dpi). Therefore, this control experiment showed that GXM accumulation in tissues and serum during a typical intratracheal infection is comparable to previous reports in the field of cryptococcosis. These measurements validated that GXM delivered into the nasal cavity can be detected in tissues other than the lungs, causes inflammation of the pulmonary tissue, and its concentrations reach sub-physiological levels in tissue and serum.

TABLE 1
GXM levels in tissues of C57BL/6 mice (n = 5)
24 h post-intranasal administration.

| Tissue | GXM concentration* | |
|---|---|---|
| Brain | 0.21 ± 0.13 | μg/g |
| Lung | 2.17 ± 0.09 | μg/g |
| Serum | 0.34 ± 0.05 | μg/mL |

*The background levels of the ELISA readings for the organs and sera excised and withdrawn, respectively, from the negative control mice (no GXM administration) were on average 0.06 (background). GXM was undetected in these samples.

### Example 4. Pre-Conditioning with GXM Exacerbates Murine Cryptococcosis and Mortality

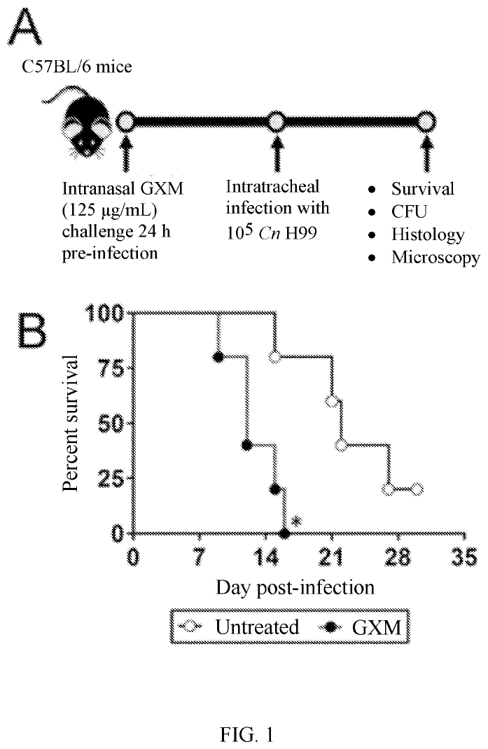

We investigated the impact of GXM prior to C57BL/6 infection with C. neoformans H99 strain cells (FIG. 1). Mice were treated intranasally with 125 μg/mL of GXM 24 h pre-inoculation with 10⁵ C. neoformans yeast cells (FIG. 1 Panel A). Cryptococcosis progression was compared between these mice and rodents infected with the fungus only (untreated). Intranasal administration of GXM significantly accelerated the death of C. neoformans-infected mice relative to control mice (n=10 mice per group; P<0.001; FIG. 1 Panel B). On day 16 post-infection, 100% of GXM-treated mice had died compared to 20% of untreated mice. On average, GXM-treated mice died 12-days post-infection, whereas untreated mice died 22-days post-infection (FIG. 1 Panel B). These findings indicate that intranasal GXM sensitization enhances mortality in mice upon pulmonary infection.

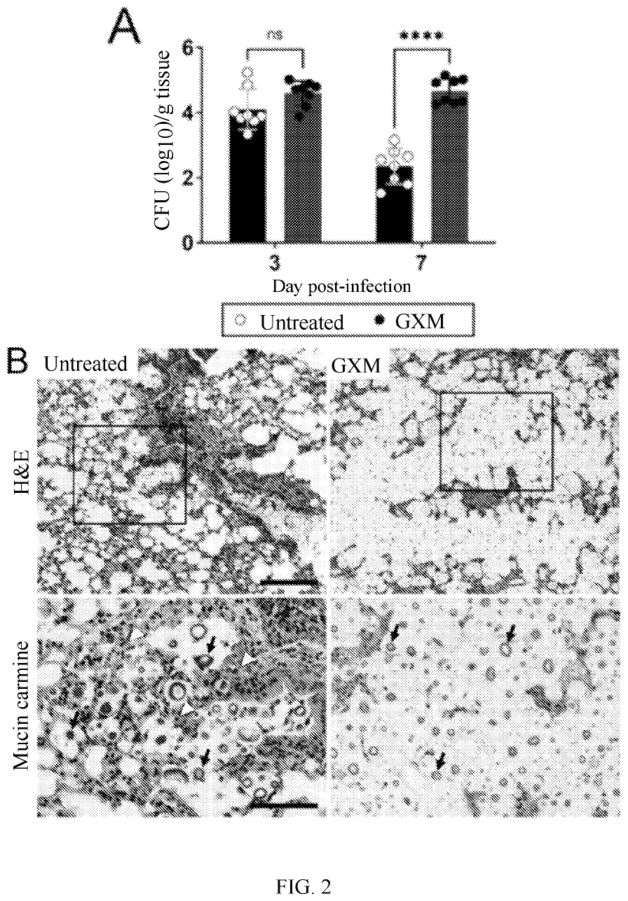

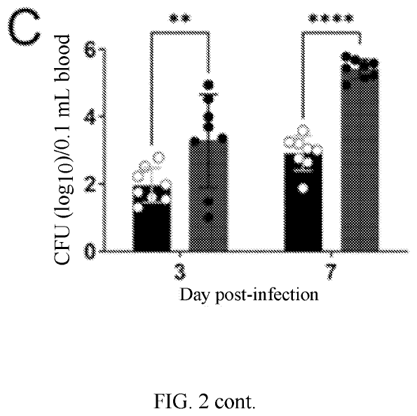

We quantified C. neoformans pulmonary burden in untreated and GXM-treated mice 3- and 7-days post-infection. Although we did not observe cryptococcal load differences between untreated (4.1×10⁴ colony forming units (CFU)) and GXM (4.59×10⁴ CFU)-challenged rodents 3-dpi (FIG. 2 Panel A), we demonstrated that the difference in pulmonary fungal burden was evident 7-dpi, with GXM-treated animals (4.66×10⁴ CFU) having a significantly higher burden than untreated mice (2.35×10² CFU; P<0.0001; FIG. 2 Panel A). Interestingly, untreated mice infected with cryptococci showed a considerable reduction in fungal load 7-dpi (4.1×10⁴ CFU) relative to 3-dpi (2.35×10² CFU). Hematoxylin and eosin (H&E) staining of pulmonary tissue removed from untreated mice 7-dpi evinced localized inflammation and dense cellular infiltration around aggregates or individual cryptococci (FIG. 2 Panel B; left upper panel; 20×). Mucine carmine staining of magnified lung tissue from untreated rodents showed C. neoformans cells of diverse sizes surrounded by inflammatory cells with limited or contained capsular polysaccharide production particularly around individual aggregates or individual cryptococci (FIG. 2 Panel B; left lower panel; 40×). In contrast, lungs excised from GXM-treated mice exhibited high fungal burden characterized by large numbers of cryptococci embedded in abundant amounts of capsular polysaccharide with little inflammation (FIG. 2 Panel B; right upper (20×) and lower (40×) panels). In addition, Cryptococci were more extensively found in the blood and brain of GXM-treated mice when compared to the untreated group (FIG. 2 Panel C). These results indicate that GXM dysregulates host homeostasis and enhances disease by increasing pulmonary fungal burden, inhibiting host inflammation, facilitating the access of cryptococci to the bloodstream, and enhancing fungal dissemination to the CNS.

### Example 5. GXM Facilitates C. neoformans Dissemination from the Lungs to the CNS

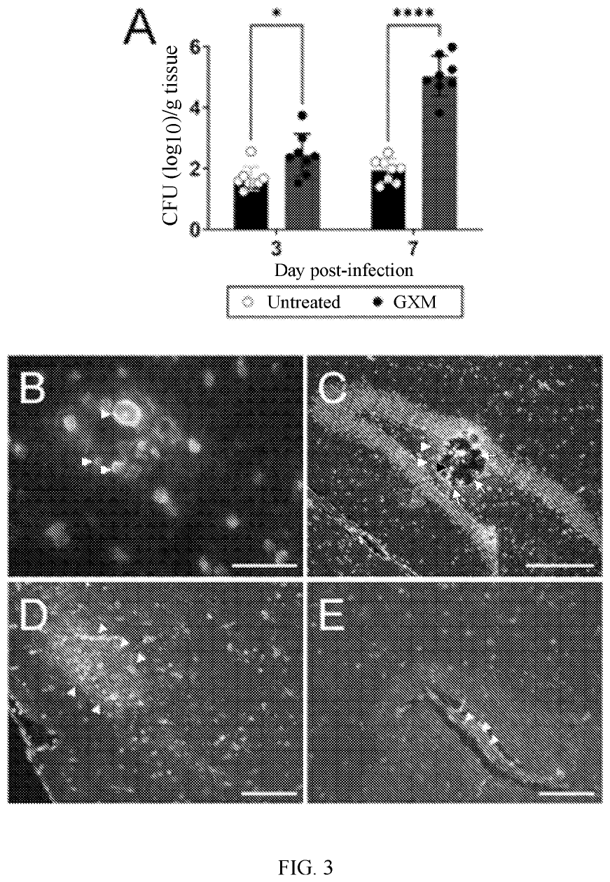

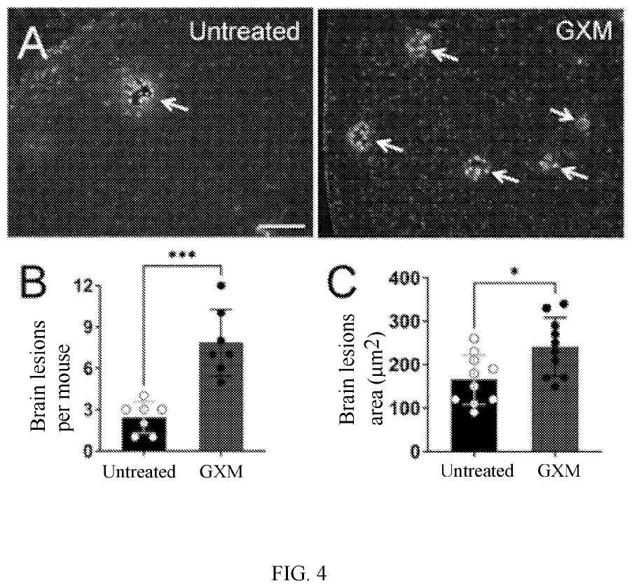

We investigated the impact of exogenous GXM administration on C. neoformans dissemination to the brain (FIG. 3). We demonstrated that brain fungal load in GXM-treated animals (3-dpi, 2.45×10² CFU; 7-dpi, 5.03×10⁵ CFU) were significantly higher than in untreated (3-dpi, 1.67×10¹ CFU; 7-dpi, 1.94×10¹ CFU) mice (3-dpi, P<0.05; 7-dpi, P<0.0001; FIG. 3 Panel A). Immunofluorescence (IF) staining and confocal microscopy of C. neoformans-infected brain tissue 7-dpi showed cryptococci of different size embedded in neuronal tissue (FIG. 3 Panel B). A tissue section of the dentate gyrus in the hippocampus of a GXM-challenged mouse exhibited a large cryptococcoma filled with yeasts cells and substantial amounts of capsular GXM released (FIG. 2 Panel C). Extensive accumulation of GXM (white arrowheads) was also observed in cortical tissue (FIG. 3 Panel D) and a large blood vessel (FIG. 3 Panel E) in the brain parenchyma of GXM-treated and C. neoformans-infected mice. In addition, immunofluorescence staining showed a significantly higher number of lesions in the brains of GXM-treated mice (average, 7.86±0.91) than those of untreated animals (2.43±0.43) (P<0.001) (FIG. 4 Panels A-B). The average areas of brain lesions of GXM-treated infected mice reached 241 μm²±27.79, whereas lesions of control mice average 166 μm²±75 (P<0.05) (FIG. 4 Panel C). These studies demonstrate that isolated GXM administration increases the permeability of the CNS to C. neoformans invasion.

### Example 6. GXM Reduces the Expression of Tight Junction Proteins in Mouse Brain Tissue

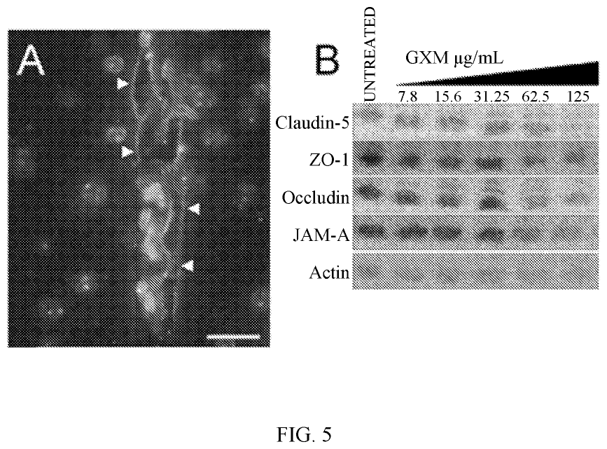

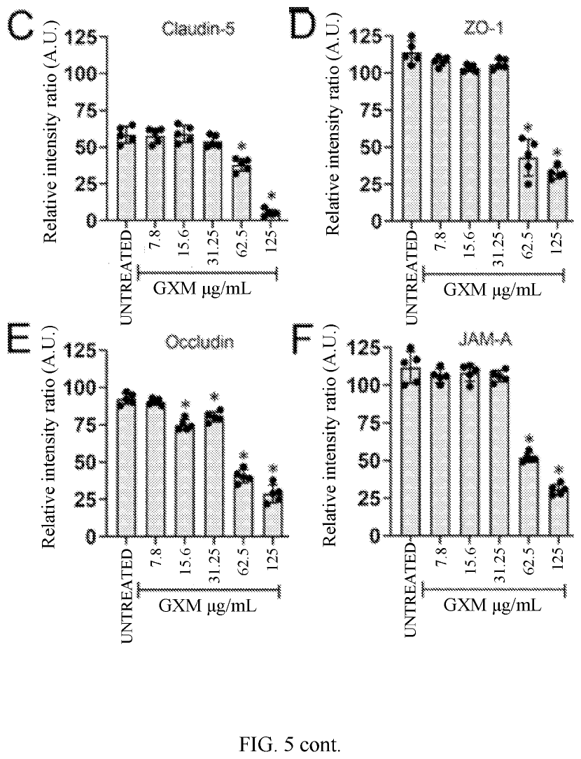

To confirm that GXM reached the CNS of challenged uninfected mice (7.8 to 250 μg/mL; PBS-instilled animals were used as controls, untreated) 24 h after intranasal administration, immunofluorescence staining using GXM-specific monoclonal Ab (mAb) 18B7 demonstrated fungal GXM distribution throughout the brain parenchyma 24 h after administration (FIG. 5 Panel A). During pathologic conditions, tight junction disruptions promote enhanced leukocyte adhesion to, and transmigration across, CNS vessels, resulting in BBB permeability and parenchymal leukocyte accumulation. Tight junction proteins provide essential structural support to the BBB, playing an important role in maintaining a safe neural microenvironment in the brain. Using western blot analysis (FIG. 5 Panel B), we found that GXM administration prior to C. neoformans pulmonary infection altered specific tight junction expression in the mouse brain. GXM doses ≥62.5 μg/mL decreased the expression of claudin-5 (FIG. 5 Panel C), ZO-1 (FIG. 5 Panel D), and JAM-A (FIG. 5 Panel F; P<0.05) in brain tissue. Interestingly, occludin downregulation was observed in rodent brains challenged with GXM doses ≥15.6 μg/mL (FIG. 5 Panel E). Our data indicate that GXM-induced tight junction protein reduction may cause brain alterations including an increase in susceptibility of the CNS to cryptococcal infection.

### Example 7. GXM Alters the Distribution/Intensity of Tight Junctions (TJs) on Human Brain Endothelial Cells (HBECs)

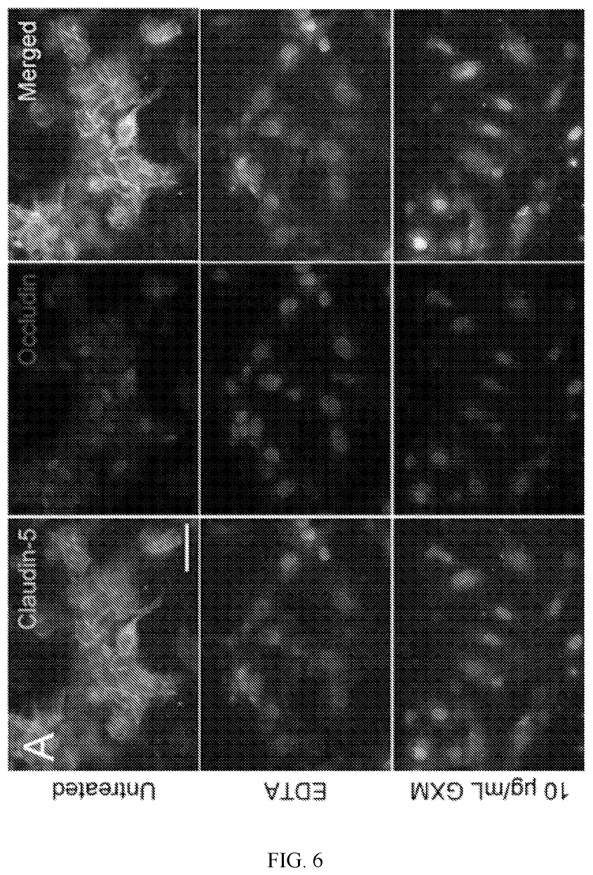

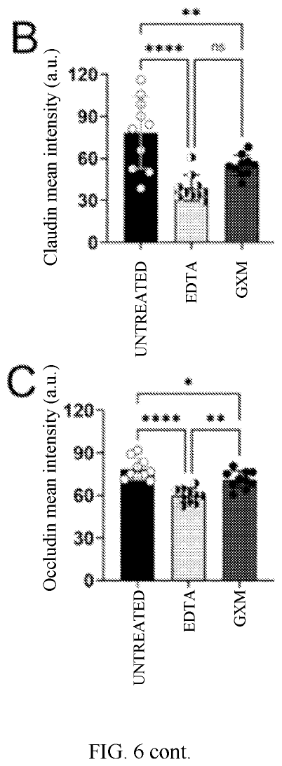

To validate the results obtained in the murine model and to determine the impact of GXM on tight junctions, we analyzed the distribution of tight junctions (claudin-5 and occludin) in HBECs after exposure to the GXM for 4 h using immunofluorescence microscopy (FIG. 6). The distribution of claudin and occludin on HBECs was considerably reduced after incubation with 10 μg/mL of GXM (FIG. 6 Panel A). EDTA (10 μg/mL) was used as a positive control and, similarly to GXM, substantially decreased the distribution of tight junctions on the surface of HBECs. Quantification of claudin-5 intensity on HBECs using the NIH ImageJ software demonstrated that GXM (P<0.01) and EDTA (P<0.0001) significantly reduced this tight junction intensity relative to untreated control HBECs (FIG. 6 Panel B). There were no differences in the distribution of claudin-5 in HBECs incubated with EDTA or GXM. Occludin intensity was also significantly decreased in HBECs cultured with GXM (P<0.05) or EDTA (P<0.0001) compared to untreated cells (FIG. 6 Panel C). Similarly, EDTA-treated HBECs evinced lower occludin intensity on their surfaces than GXM-treated cells (P<0.01). These experiments indicate that the distribution/intensity of tight junctions on HBECs is reduced after incubation with GXM.

### Example 8. GXM Increases the Expression of RhoA Cytoskeletal Regulator in a Time-Dependent Manner

To investigate the disruption of BBB integrity by GXM, we measured the activation of RhoA, a member of the Ras-related small GTPase Rho family that signals to regulate the actin cytoskeleton. When bound to GTP, RhoA functions as a regulatory switch that confers increased endothelial cell permeability and BBB disruption.

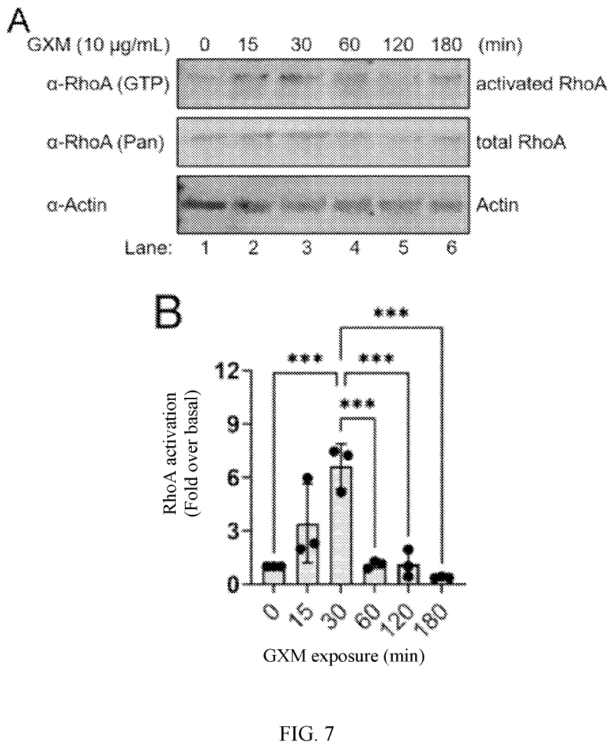

To determine whether RhoA is activated during GXM-mediated disruption of tight junction integrity, levels of both RhoA (pan) and RhoA-GTP (activated form) were measured in lysates of HBECs by western blot analysis (FIG. 7). HBECs (n=3) were plated in 24-well plates and grown to confluency prior to stimulation with 10 μg/mL of GXM. RhoA activation was measured by 0, 15, 30, 60, 120, and 180 min (FIG. 7 Panel A). RhoA activation was induced immediately upon stimulation by GXM, reaching a peak 6-fold increase after 30 min (FIG. 7 Panel B). Interestingly, RhoA activation dropped to near-basal levels after 60 min before decreasing below basal levels at 180 min. RhoA activation is associated with increased cell permeability and actin cytoskeletal fiber formation, and its involvement in modulating tight junction integrity is of interest during cryptococcal meningoencephalitis. Actin was used as a control, exhibiting a decrease in expression over the course of the time points indicated and providing support for the role of GXM in modulating cytoskeletal properties. Our data suggest that GXM-induced RhoA activation at 30 min, followed by a prominent decrease after 60 min.

RhoA signals through ROCK 1 (Rho associated coiled-coil containing protein kinase 1 to inhibit myosin-light chain phosphatase which in turn leads to stable phosphorylation and activation of myosin-light chain (MLC). Activated MLC mediates actin stress fiber formation during endothelial barrier permeability. We observed a 10-fold increase in MLC phosphorylation after 15 min of GXM stimulation, concurrent with activation of RhoA. Interestingly, phosphorylation of MLC remained elevated after 120 min, indicating that actin remodeling was prolonged after initial RhoA stimulation. To assess the extent of actin remodeling in HBECs treated with GXM, we performed fluorescence confocal microscopy on cells stained with the fluorescent actin dye phalloidin and treated with GXM over time (0, 15, 30, 60, 120 and 180 min). Although the levels of actin expression did not significantly change in response to GXM treatment, substantial changes were observed in the actin cytoskeleton following GXM stimulation. In untreated confluent HBEC monolayers (0 min), actin was localized to the periphery of cells. Actin stress fibers begin to form after 15 min of GXM stimulation and continued to develop over time. In addition, actin remodeled away from the cell periphery to adopt a perinuclear localization after 60 min, which persisted throughout the rest of the experiment. Disruption of the endothelial monolayer was observed as early as 15 min, but was most prominent at 60, 120 and 180 min. These results are consistent with the time course for MLC phosphorylation in response to GXM. Our data indicate that GXM-induced RhoA activation and RhoA-mediated actin cytoskeleton remodeling. Without wishing to be bound by theory, it is possible that RhoA-mediated actin cytoskeleton remodeling may be mechanistically important GXM-mediated permeability of the BBB.

GXM-induced RhoA signaling to myosin-light chain (MLC) was significant and time-dependent, and demonstrated morphological changes in the actin cytoskeleton of cells treated with GXM. These observations indicate that GXM induces remodeling of the actin cytoskeleton in endothelial cells.

### Example 9. GXM Disrupts the Trans-Endothelial Electrical Resistance (TEER) of HBECs in a Blood Brain Barrier (BBB) Model and Enhances its Permeability

The TEER is a widely accepted quantitative technique to measure the integrity of tight junction dynamics in cell culture models of endothelial monolayers. Since GXM reduces the expression (in vivo) and distribution/intensity (in vitro) of tight junctions, we investigated the impact of the capsular polysaccharide on TEER of an in vitro BBB model that consisted of HBECs and pericytes (FIG. 8 Panels A-B). Pericytes are cells present at intervals along the walls of blood vessels. In the CNS, they are important for blood vessel formation, maintenance of the BBB, regulation of immune cell entry to the CNS, and control of brain blood flow. We investigated single (HBECs) and dual (HBECs+pericytes) in vitro BBB models. The BBB model consisting of HBECs+pericytes demonstrated a significantly higher TEER (2-fold) than the system with only HBECs (P<0.01), which indicated that pericytes substantially strengthen the interactions between HBECs. Using this model, all the BBBs treated with GXM or EDTA demonstrated a significant drop in TEER (~60% or more) 1 h post-exposure relative to the untreated BBBs (P<0.05; FIG. 8 Panel B). EDTA-treated BBBs demonstrated a lower TEER percentage than BBBs incubated with 50 (P<0.05) and 100 (P<0.01) μg/mL of GXM. Also, BBBs cultured with 100 μg/mL of GXM exhibited lower TEER percentage than BBBs exposed to 10 μg/mL of the fungal polysaccharide (P<0.01). BBBs treated with 10 μg/mL of GXM maintained ~60% TEER percentage reduction after 1 h compared to the control BBBs. Notably, all the other conditions demonstrated a 100% TEER or tight junction integrity reduction in BBBs after 1 h. To better understand the minimal GXM concentration required for compromising the integrity of the in vitro BBB model, we performed a dose response curve exposing cells to 0.001 to 100 μg/mL of GXM. We calculated that the inhibitory concentration (IC50) needed to make the system 50% permeable was 9.95 μg/mL of GXM. Next, we assessed the BBB permeability to streptavidin-horseradish peroxidase (HRP) after 1 h treatment with GXM or EDTA (FIG. 8 Panels C-D). All the GXM (10 μg/mL, P<0.01; 100 μg/mL, P<0.001)- or EDTA (P<0.001)-treated HBECs exhibited significantly more permeability than the untreated cells (FIG. 8 Panel D). HBECs cultured with concentrations ≥50 μg/mL of GXM or 10 μg/mL of EDTA displayed similar increased barrier permeability to streptavidin-HRP. HBECs incubated with 10 μg/mL GXM demonstrated considerably lower barrier permeability than EDTA-treated (P<0.05) cells. The results demonstrate that GXM disturbs the TEER and integrity of HBEC-closed interactions in a BBB in vitro model.

Given that GXM inhibits the expression of tight junctionss and alters the TEER in HBECs, we investigated the permeability of HBECs to C. neoformans strain H99 cell transmigration after pre-incubation with 10 μg/mL of GXM of EDTA for 1 h (FIG. 9 Panel A). GXM-(P<0.01) and EDTA-(P<0.0001) treated HBECs demonstrated significant passage of cryptococci compared to untreated cells (FIG. 9 Panel B). Our studies indicate that GXM promotes HBEC disruption and C. neoformans penetration.

### Example 10. GXM Stimulates Vasodilation of Blood Vessels Via Endothelium-Dependent Mechanisms

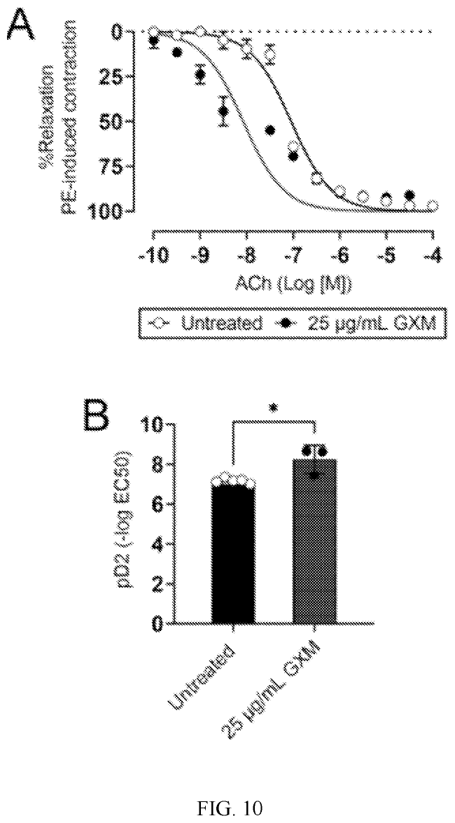

To understand the influence of GXM on endothelial cells of blood vessels, mouse superior mesenteric arteries (SMA) were isolated, cultured in absence or presence of 25 μg/mL of GXM ex vivo for 40 min, and evaluated for vascular reactivity using a wire myograph in the presence of acetylcholine (ACh; to test for endothelium-dependent vasodilation) or phenylephrine (PE; to test for vasoconstriction) (FIG. 10). SMA incubated with the GXM were less responsive to PE and decreased their contraction relative to untreated arteries (n=5 per group; FIG. 10 Panel A). However, arteries incubated with 25 μg/mL of GXM were substantially more sensitive to ACh and significantly increased their vasodilation compared to untreated blood vessels (FIG. 10 Panel A). GXM-treated SMA (n=3) were significantly more sensitive to ACh than untreated arteries (n=5; P<0.05; FIG. 10 Panel B). The results indicate that GXM promotes blood vessel relaxation by endothelium-dependent mechanisms.

### Example 11

We found that intranasal GXM instillation (concentrations: 15.6-125 μg/mL) alone disrupted tight junction and adhesion proteins in rodents, validating the importance of isolated GXM in destabilizing the BBB. Isolated GXM also impacted tight junctions in endothelial cells. Claudin-5, ZO-1, and JAM-A were downregulated at concentrations ≥62.5 μg/mL, whereas occludin seemed to be more susceptible to GXM and was reduced at concentrations ≥15.6 μg/mL. Western blot analyses in murine brain extracts 24 h after GXM sensitization and IF in HBECs cultured with the capsular polysaccharide for 4 h demonstrated a significant reduction in the expression/distribution of claudin-5 and occludin. These integral proteins are located in the apical region of the cell and act as gatekeepers for BBB paracellular passage. Moreover, the in vivo GXM sensitization studies showed that the expression of actin, a housekeeping protein that forms microfilaments in the cytoskeleton and has intimate interactions with cellular membranes was also affected.

To rule out the possibility that GXM instillation may damage BBB's endothelial cells in mice due to high concentration overload, we have tested GXM instillation in mice up to 500 μg/mL and tight junction expression increases at that concentration indicating tight junction expression can be recovered. The same pattern was observed in the actin bands. Thus, the inhibition of intercellular junctions and actin is transient.

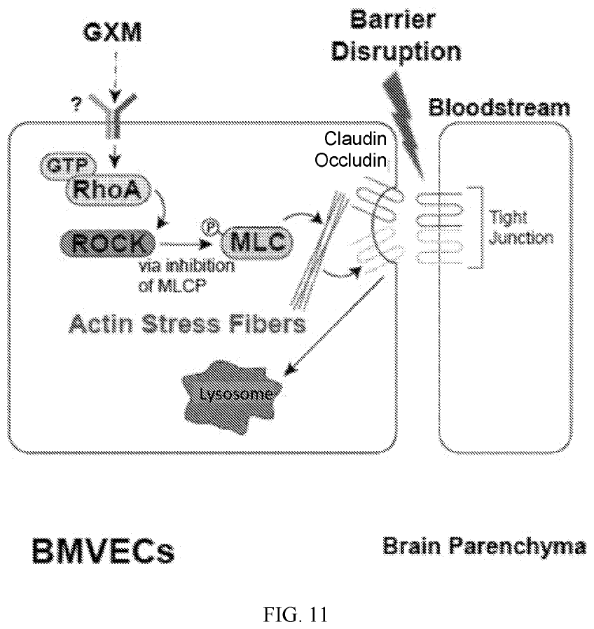

Our findings of GXM-induced RhoA activation substantiates the impact of this capsular polysaccharide on the BBB structural integrity. We demonstrated that GXM may regulate BBB transmigration by causing rapid structural changes in the BBB including weakening of tight junction and disruption of HBEC interactions (FIG. 11). GXM stimulates activation of RhoA and MLC phosphorylation and causes actin stress fiber formation. Actin stress fibers may contribute to the internalization and lysosomal degradation of claudin and occludin, thereby disrupting TJs between brain microvascular endothelial cells.

We demonstrated GXM-treated HBECs grown in transwells displayed higher permeability and passage to cryptococci relative to the untreated and EDTA-treated HBEC conditions. Exposure of HBECs to GXM resulted in a significant reduction of tight junction distribution on their surface. GXM addition to the in vitro BBB model caused a dramatic reduction in the BBB integrity and TEER. In contrast, there was no defect when adding cryptococci, which also produce and shed GXM in the medium.

We demonstrated that exogenous isolated GXM administration increases permeability of the BBB and facilitates CNS delivery.

### Example 12. GXM Induces Remodeling of the Actin Cytoskeleton and VE-Cadherin in Human ECs

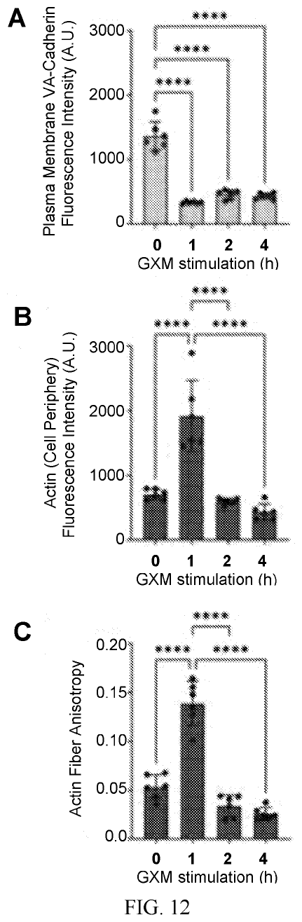

Endothelial barrier stability is maintained by VE-cadherin, an important transmembrane protein expressed within adherens junctions. To investigate the impact of GXM exposure on VE-cadherin, confluent monolayers of human ECs were incubated with 10 μg/mL GXM, then fixed and labeled with anti-VE-Cadherin antibodies and the fluorescent actin dye phalloidin-549 and visualized with confocal microscopy. In untreated monolayers, endogenous VE-cadherin localized to the plasma membrane, however exposure to GXM for 1 h resulted in separation of cell-cell contacts, internalization of VE-cadherin and polymerization of actin into stress fibers. Prolonged exposure to GXM (2-4 h) caused cells to separate further, concurrent with the internalization and accumulation of VE-cadherin into internal punctae. Internalization of VE-cadherin was quantified by measuring fluorescence intensity at the cell periphery using line-scan analysis (P<0.0001, n=6 measurements per time point; FIG. 12A). Interestingly, we observed transient actin stress-fiber formation) after 1 h of GXM exposure, quantified by line-scan analysis of the cell periphery near the plasma membrane (P<0.0001, n=6 measurements per time point; FIG. 12B). Furthermore, we used FibrilTool to analyze the organization of actin into stress fibers in the cytoplasm and membranes of ECs treated with GXM. Anisotropy represents a measure of how organized fibers are into parallel structures. GXM stimulation increased anisotropy of F-Actin almost three-fold compared to F-Actin in unstimulated cells (P<0.0001, n=6 measurements per time point; FIG. 12C). These observations indicate that GXM stimulates rapid remodeling of endothelial adherens junctions and the actin cytoskeleton to facilitate endothelial barrier disruption.

1. A method of increasing delivering a molecule to the central nervous system (CNS) of a subject comprising administering the molecule to the subject in combination with isolated glucuronoxylomannan (GXM).

2. The method of claim 1, wherein the isolated GXM is administered intranasally, or intravascularly.

3. The method of claim 1 or 2, wherein the molecule is administered intranasally, intravascularly, or subcutaneously.

4. The method of any one of claims 1-3, wherein the isolated GXM is administered at a concentration of about 10 μg/mL to about 200 μg/mL.

5. The method of any one of claims 1-4, wherein the molecule is a therapeutic molecule.

6. The method of claim 5, wherein the molecule is a protein, an antibody, an antibody fragment, or a small molecule drug.

7. The method of any one of claims 1-6, wherein delivering the molecule to the CNS comprises delivering the molecule to the brain.

8. The method of any one of claims 1-7, wherein the subject has a neurological disease, a CNS cancer, or a CNS infection.

9. The method of any one of claims 1-8, wherein the isolated GXM is isolated from C. neoformans.

10. A method of increasing permeability of the blood brain barrier in a subject comprising administering to the subject isolated GXM.

11. The method of claim 10, wherein the isolated GXM is administered intranasally, or intravascularly.

12. The method of claim 10 or 11, wherein the isolated GXM is administered at a concentration of about 10 μg/mL to about 200 μg/mL.

13. The method of any one of claims 10-12, wherein the isolated GXM is isolated from C. neoformans.

14. The method of any one of claims 10-13, wherein the permeability of the blood brain barrier is increased transiently.

15. A composition for delivery of a molecule to the CNS comprising the molecule and a pharmaceutically effective dose of isolated GXM.

16. The composition of claim 15, wherein the molecule is a therapeutic molecule.

17. The composition of claim 15, wherein the molecule is a protein, an antibody, an antibody fragment, a biologic agent, or a small molecule drug.

18. The composition of any one of claims 15-17, wherein the isolated GXM is isolated from C. neoformans.

19. The composition of any one of claims 15-18, wherein the composition is formulated for intranasal or intravascular administration.

20. A pharmaceutical combination comprising a therapeutic molecule and an isolated GXM.

21. The pharmaceutical combination of claim 20, wherein the molecule is a protein, an antibody, an antibody fragment, or a small molecule drug.

22. The pharmaceutical combination of claim 20 or 21, wherein the isolated GXM is isolated from C. neoformans.

23. The pharmaceutical combination of any one of claims 20-22, wherein the therapeutic molecule and an isolated GXM are formulated for administration to a subject separately.

24. The pharmaceutical combination of claim 23, wherein the isolated GXM is formulated for intranasal or intravascular administration.
